(19) United States
(12) Patent Application Publication
(10) Pub. No.: US 2025/0257339 A1
(43) Pub. Date: Aug. 14, 2025

(54) AFFINITY CHROMATOGRAPHY USING AN ELUTION BUFFER COMPRISING GLYCINE AND ARGININE

(71) Applicant: Takeda Pharmaceutical Company Limited, Osaka-shi (JP)

(72) Inventor: Wei LU, Lexington, MA (US)

(21) Appl. No.: 19/010,369

(22) Filed: Jan. 6, 2025

Related U.S. Application Data
(63) Continuation of application No. PCT/IB2023/057035, filed on Jul. 7, 2023.
(60) Provisional application No. 63/359,120, filed on Jul. 7, 2022.

(51) Int. Cl. C12N 9/16; B01D 15/18; B01D 15/36; B01D 15/38; B01D 15/42

(52) U.S. Cl. CPC C12N 9/16; B01D 15/1871; B01D 15/363; B01D 15/3809; B01D 15/426; C12Y 301/06001; C12Y 301/06013

(57) ABSTRACT

The present invention provides, among other things, methods of purifying a protein or polypeptide by affinity chromatography, for example, custom affinity chromatography, followed by downstream chromatographic steps, comprising eluting the recombinant protein from the affinity column using an elution buffer wherein the elution buffer comprises glycine and arginine at a concentration such that conductivity is no greater than the conductivity limit of the subsequent chromatographic resin.

### CROSS-REFERENCE TO RELATED APPLICATIONS

This application is a Continuation Application of International Application No. PCT/IB2023/057035, filed on Jul. 7, 2023, which claims priority to, and the benefit of U.S. Provisional Patent Application No. 63/359,120, filed on Jul. 7, 2022, the contents of all of which are incorporated by reference herein in their entirety for all purposes.

### BACKGROUND

Purification of recombinant proteins and polypeptides has several applications in therapeutics. Typically, a variety of chromatographic steps are included in protein purification processes including affinity chromatography, ion exchange chromatography, mixed mode chromatography, and/or hydrophobic interaction chromatography, among others. Increasing yield and maintaining high activity is a challenge for recombinant protein purification processes. It remains a challenge to tailor purification processes with multiple steps for efficient and high yield purification at each step while maintaining compatible elution and pH conditions that favor downstream processing. For example, while acidic elution is commonly used in affinity chromatography, some proteins are not very stable under acidic pH.

### SUMMARY OF THE INVENTION

The present invention provides, among other things, improved methods of purifying protein or polypeptide by affinity chromatography (e.g., custom affinity chromatography), comprising eluting a polypeptide from an affinity chromatography column using an elution buffer with pH and conductivity properties that are compatible with further downstream purification and processing, thereby minimizing loss of product and product quality. In one aspect, the elution buffer comprises glycine and arginine. The present invention also provides a platform technology to optimize the elution buffer for affinity chromatography (e.g., custom affinity chromatography). In one aspect, the platform technology can provide specific ranges of the concentration of glycine and arginine, conductivity, and/or pH for the elution buffer.

The inventors of the present application surprisingly and unexpectedly found that affinity resins have an ion exchange effect at the start of elution, causing subsequent conductivity and pH changes on the affinity column, thereby reducing product quality. For example, as the elution buffer contacts the resin, the resin is acidified, and has an anion exchange effect, thereby negatively charged ions bind the resin and are depleted from the solution. The inventors discovered that multiple affinity resins had an ion exchange effect during elution.

Further, acidic elution buffers commonly used in affinity chromatography cause denaturation of some proteins, leading to reduced yield and activity. Formation of aggregates leading to precipitation and reduced yield is another problem. There are different strategies to increase the elution pH from a low pH to a milder pH, including, for example, increasing salt concentration or adding organic solvents. However, these have disadvantages, for example, high salt concentration generates high conductivity, limiting the choice of next purification or polishing step, while organic solvents denature protein, have high viscosity and have safety issues.

Through careful experiments, inventors of the present application have designed affinity elution buffers, for example, comprising arginine and glycine, having compatible pH and conductivity properties with downstream chromatographic steps, and leading to increased protein stability. The inventors of the present application have obtained, for example, affinity elution pools with low conductivity allowing flexibility in choice of downstream chromatographic steps. For example, if hydrophobic interaction chromatography (HIC) is used downstream, the conductivity of the elution pool is appropriately increased, for example, by adding salt, so that it is compatible with the HIC column. If the affinity elution pool has a high conductivity, some downstream chromatographic steps such as ion exchange chromatography require extensive dilution which increases costs. On the other hand, higher elution pool conductivity is compatible with hydrophobic interaction chromatography or mixed mode chromatography, and suitable arginine and glycine buffer combinations are selected to adapt to the next downstream chromatographic column. Amino acids such as arginine and glycine are safe excipients, soluble in aqueous solvents, and increase protein stability by facilitating preferential interaction of solvent components with proteins, and through beneficial effects on surface tension and structure of an aqueous solvent.

In some embodiments, arginine and glycine are added to the elution buffer to prevent lowering of conductivity. For example, in an exemplary purification of arylsulfatase A, when a glycine hydrochloride elution buffer is used, negatively charged chloride ions are depleted and the remaining protons and zwitterions have no conductivity. Addition of arginine hydrochloride or sodium chloride prevents the conductivity from dropping too low. Without wishing to be bound by any particular theory, it is contemplated that the resin cannot deplete cations, and the addition of cations to the elution buffer prevents product loss. In some embodiments, the addition of cations (e.g., arginine) to glycine buffer prevents drop in conductivity which benefits product quality.

In some embodiments, arginine and glycine are added to the elution buffer to prevent lowering of pH or pH instability. In some embodiments, for example, in purifying arylsulfatase A, when acetate is used as an elution buffer, the acetate anion is depleted by the resin, which causes more acidic radicals, decreasing pH. The resultant pH instability affects product quality. In some embodiments, the addition of cations (e.g., arginine) stabilizes elution pH.

In one aspect, provided herein is a method of purifying a polypeptide, comprising loading the polypeptide onto an affinity chromatography column, eluting the polypeptide from the affinity chromatography column using an elution buffer comprising glycine and arginine, subjecting the eluate from the affinity chromatography column to a subsequent chromatography column comprising a resin, wherein conductivity of the elution buffer is no greater than conductivity limit of the resin in the subsequent chromatography column.

In one aspect, provided herein is a method of purifying a polypeptide, comprising loading the polypeptide onto an affinity chromatography column, eluting the polypeptide from the affinity chromatography column using an elution buffer comprising glycine and arginine, wherein conductivity of the elution buffer is no greater than 30 mS/cm. In some embodiments, conductivity of the elution buffer is no greater than 5, 10, 15, 20, 25 or 30 mS/cm. In some embodiments, conductivity of the elution buffer is no greater than 8, 10, 12, 14, 16, 18, 20, 24, 26, 28, 30 mS/cm. In some embodiments, conductivity of the elution buffer is about 5-10 mS/cm, 6-10 mS/cm, 7-10 mS/cm, 8-10 mS/cm, or 9-10 mS/cm. In some embodiments, conductivity of the elution buffer is about 5-30 mS/cm, 10-30 mS/cm, 15-30 mS/cm, 20-30 mS/cm, or 25-30 mS/cm.

In some embodiments, provided herein is a method, wherein the elution buffer comprises glycine at a concentration no greater than 1500 mM, and arginine at a concentration no greater than 300 mM. In some embodiments, provided herein is a method, wherein the elution buffer comprises glycine at a concentration no greater than 50 mM, 60 mM, 70 mM, 80 mM, 90 mM, 100 mM, 200 mM, 300 mM, 400 mM, 500 mM, 600 mM, 700 mM, 800 mM, 900 mM, 1000 mM, 1100 mM, 1200 mM, 1300 mM, 1400 mM or 1500 mM and arginine at a concentration no greater than 100 mM, 150 mM, 200 mM, 250 mM, 300 mM. In some embodiments, provided herein is a method, wherein the elution buffer comprises glycine at a concentration of X mM and arginine at a concentration of Y mM, wherein the X and Y satisfy an inequality 0.02X+0.1Y≤30. In some embodiments, provided herein is a method, wherein the elution buffer comprises glycine at a concentration of X mM and arginine at a concentration of Y mM, wherein the X and Y satisfy an inequality 0<0.02X+0.1Y≤30. In some embodiments, provided herein is a method, wherein the elution buffer comprises glycine at a concentration of X mM and arginine at a concentration of Y mM, wherein the X and Y satisfy an inequality 1<0.02X+0.1Y≤30. In some embodiments, provided herein is a method, wherein the elution buffer comprises glycine at a concentration of X mM and arginine at a concentration of Y mM, wherein the X and Y satisfy an inequality 2<0.02X+0.1Y≤30. In some embodiments, provided herein is a method, wherein the elution buffer comprises glycine at a concentration of X mM and arginine at a concentration of Y mM, wherein the X and Y satisfy an inequality 3<0.02X+0.1Y≤30. In some embodiments, provided herein is a method, wherein the elution buffer comprises glycine at a concentration of X mM and arginine at a concentration of Y mM, wherein the X and Y satisfy an inequality 4<0.02X+0.1Y≤30. In some embodiments, provided herein is a method, wherein the elution buffer comprises glycine at a concentration of X mM and arginine at a concentration of Y mM, wherein the X and Y satisfy an inequality 5<0.02X+0.1Y≤30. In some embodiments, provided herein is a method, wherein the elution buffer comprises glycine at a concentration of X mM and arginine at a concentration of Y mM, wherein the X and Y satisfy an inequality 10<0.02X+0.1Y≤30. In some embodiments, provided herein is a method, wherein the elution buffer comprises glycine at a concentration of X mM and arginine at a concentration of Y mM, wherein the X and Y satisfy an inequality 15<0.02X+0.1Y≤30. In some embodiments, provided herein is a method, wherein the elution buffer comprises glycine at a concentration of X mM and arginine at a concentration of Y mM, wherein the X and Y satisfy an inequality 20<0.02X+0.1Y≤ 30. In some embodiments, provided herein is a method, wherein the elution buffer comprises glycine at a concentration of X mM and arginine at a concentration of Y mM, wherein the X and Y satisfy an inequality 25<0.02X+0.1Y≤30.

In some embodiments, provided herein is a method, wherein the elution buffer comprises glycine at a concentration of no greater than 500 mM, arginine at a concentration of no greater than 100 mM. In some embodiments, provided herein is a method, wherein the elution buffer comprises glycine at a concentration of no greater than 50 mM, 100 mM, 200 mM, 300 mM, 400 mM or 500 mM, arginine at a concentration of no greater than 10 mM, 20 mM, 30 mM, 40 mM, 50 mM, 60 mM, 70 mM, 80 mM, 90 mM or 100 mM. In some embodiments, provided herein is a method, wherein the elution buffer comprises glycine at a concentration of X mM and arginine at a concentration of Y mM, wherein the X and Y satisfy an inequality 0.02X+0.1Y≤10. In some embodiments, provided herein is a method, wherein the elution buffer comprises glycine at a concentration of X mM and arginine at a concentration of Y mM, wherein the X and Y satisfy an inequality 0<0.02X+0.1Y≤10. In some embodiments, provided herein is a method, wherein the elution buffer comprises glycine at a concentration of X mM and arginine at a concentration of Y mM, wherein the X and Y satisfy an inequality 1<0.02X+0.1Y≤10. In some embodiments, provided herein is a method, wherein the elution buffer comprises glycine at a concentration of X mM and arginine at a concentration of Y mM, wherein the X and Y satisfy an inequality 2<0.02X+0.1Y≤10. In some embodiments, provided herein is a method, wherein the elution buffer comprises glycine at a concentration of X mM and arginine at a concentration of Y mM, wherein the X and Y satisfy an inequality 3<0.02X+0.1Y≤10. In some embodiments, provided herein is a method, wherein the elution buffer comprises glycine at a concentration of X mM and arginine at a concentration of Y mM, wherein the X and Y satisfy an inequality 4<0.02X+0.1Y≤10. In some embodiments, provided herein is a method, wherein the elution buffer comprises glycine at a concentration of X mM and arginine at a concentration of Y mM, wherein the X and Y satisfy an inequality 5<0.02X+0.1Y≤10. In some embodiments, provided herein is a method, wherein the elution buffer comprises glycine at a concentration of X mM and arginine at a concentration of Y mM, wherein the X and Y satisfy an inequality 6<0.02X+0.1Y≤10. In some embodiments, provided herein is a method, wherein the elution buffer comprises glycine at a concentration of X mM and arginine at a concentration of Y mM, wherein the X and Y satisfy an inequality 7<0.02X+0.1Y≤10. In some embodiments, provided herein is a method, wherein the elution buffer comprises glycine at a concentration of X mM and arginine at a concentration of Y mM, wherein the X and Y satisfy an inequality 8<0.02X+0.1Y≤10. In some embodiments, provided herein is a method, wherein the elution buffer comprises glycine at a concentration of X mM and arginine at a concentration of Y mM, wherein the X and Y satisfy an inequality 9<0.02X+0.1Y≤10. In some embodiments, provided herein is a method, wherein the elution buffer comprises glycine at a concentration of no more than 500 mM and arginine at a concentration of no more than 100 mM. In some embodiments, provided herein is a method, wherein the elution buffer comprises glycine at a concentration of 10-50 mM, 50-100 mM, 100-150 mM, 150-200 mM, 200-250 mM, 250-300 mM, 300-350 mM, 350-400 mM, 400-450 mM, 450-500 mM and arginine at a concentration of 10-20 mM, 20-30 mM, 30-40 mM, 40-50 mM, 50-60 mM, 60-70 mM, 70-80 mM, 80-90 mM, 90-100 mM. In some embodiments, provided herein is a method, wherein the elution buffer comprises glycine at a concentration of 300 mM and arginine at a concentration of 40 mM.

In one aspect, provided herein is a method of purifying a polypeptide, the method comprising, loading the polypeptide onto an affinity chromatography column, eluting the polypeptide from the affinity chromatography column using an elution buffer, wherein the elution buffer comprises glycine at a concentration of no greater than 500 mM, arginine at a concentration of no greater than 100 mM.

In one aspect, provided herein is a method of purifying a polypeptide, the method comprising, loading the sample onto an affinity chromatography column, eluting the polypeptide from the affinity chromatography column using an elution buffer, wherein the elution buffer comprises glycine and arginine and the glycine and arginine are present at a ratio of at least 1:2 (molar ratio).

In some embodiments, the elution buffer of the method provided herein comprises glycine and arginine at a pH between 3.0-5.0, and the glycine and arginine are present at a ratio between 1:2 to 40:1 (molar ratio).

In some embodiments, the elution buffer comprises arginine at a concentration of about 10 mM to 300 mM. In some embodiments, the elution buffer comprises arginine at a concentration of about 10-20 mM, 20-30 mM, 30-40 mM, 40-50 mM, 50-60 mM, 60-70 mM, 70-80 mM, 80-90 mM, 90-100 mM, 100-110 mM, 110-120 mM, 120-130 mM, 130-140 mM, 140-150 mM, 150-160 mM, 160-170 mM, 170-180 mM, 180-190 mM, 190-200 mM, 200-210 mM, 210-220 mM, 220-230 mM, 230-240 mM, 240-250 mM, 250-260 mM, 260-270 mM, 270-280 mM, 280-290 mM, or 290-300 mM. In some embodiments, the elution buffer comprises arginine at a concentration of about 5 mM to 100 mM. In some embodiments, the elution buffer comprises arginine at a concentration of about 5-10 mM, 10-15 mM, 15-20 mM, 20-25 mM, 25-30 mM, 30-35 mM, 35-40 mM, 40-45 mM, 45-50 mM, 50-55 mM, 55-60 mM, 60-65 mM, 65-70 mM, 70-75 mM, 75-80 mM, 80-85 mM, 85-90 mM, 90-95 mM, or 95-100 mM.

In some embodiments, the elution buffer comprises glycine at a concentration of about 50 mM to 1500 mM. In some embodiments, the elution buffer comprises glycine at a concentration of about 50-100 mM, 100-150 mM, 150-200 mM, 200-250 mM, 250-300 mM, 300-350 mM, 350-400 mM, 400-450 mM, 450-500 mM, 500-550 mM, 550-600 mM, 600-650 mM, 650-700 mM, 700-750 mM, 750-800 mM, 800-850 mM, 850-900 mM, 900-950 mM, 950-1000 mM, 1000-1100 mM, 1100-1200 mM, 1200-1300 mM, 1300-1400 mM, 1400-1500 mM. In some embodiments, the elution buffer comprises arginine at a concentration of about 25 mM to 500 mM. In some embodiments, the elution buffer comprises arginine at a concentration of about 25-50 mM, 50-75 mM, 75-100 mM, 100-125 mM, 125-150 mM, 150-175 mM, 175-200 mM, 200-225 mM, 225-250 mM, 250-275 mM, 275-300 mM, 300-325 mM, 325-350 mM, 350-375 mM, 375-400 mM, 400-425 mM, 425-450 mM, 450-475 mM, or 475-500 mM.

In some embodiments, the method further comprises a step of adjusting the eluate from the affinity chromatography column to a pH of 4.0 to 9.0. In some embodiments, the method further comprises a step of adjusting the eluate from the affinity chromatography column to a pH of 4.0 to 5.0, 5.0-6.0, 6.0-7.0, 7.0-8.0 or 8.0-9.0.

In some embodiments, the elution buffer further comprises a step of viral inactivation of the eluate from the affinity chromatography column.

In some embodiments, the eluate of the affinity column comprises less than 5% high molecular weight aggregates (HMW). In some embodiments, the eluate of the affinity column comprises less than 4% high molecular weight aggregates (HMW). In some embodiments, the eluate of the affinity column comprises less than 3% high molecular weight aggregates (HMW). In some embodiments, the eluate of the affinity column comprises less than 2.5% high molecular weight aggregates (HMW). In some embodiments, the eluate of the affinity column comprises less than 2% high molecular weight aggregates (HMW). In some embodiments, the eluate of the affinity column comprises less than 1% high molecular weight aggregates (HMW).

In some embodiments, the affinity chromatography column uses an antibody or antigen binding fragment that specifically binds the polypeptide. In some embodiments, the affinity chromatography column uses an antibody that specifically binds the polypeptide. In some embodiments, the affinity chromatography column uses an antigen binding fragment that specifically binds the polypeptide. In some embodiments, the affinity chromatography column comprises a custom affinity chromatography resin. In some embodiments, the affinity chromatography column comprises an affinity ligand which is screened and selected from a library of ligands for the polypeptide. In some embodiments, the affinity chromatography resin comprises an affinity ligand which is screened and selected from a library of proteins or peptides for the polypeptide. In some embodiments, the affinity chromatography resin comprises an affinity ligand which is screened and selected from a library, wherein the library comprises more than 100 proteins or peptides.

In some embodiments, the method further comprises adjusting the concentration of arginine and glycine in the elution buffer based on the conductivity limit of a downstream resin for a polypeptide. In some embodiments, the method further comprises subjecting the eluate from the affinity chromatography column to a step of chromatography. In some embodiments, the method comprises subjecting the eluate from the affinity chromatography column to a step of chromatography, wherein the step of chromatography is selected from the group consisting of ion exchange, mixed mode, or hydroxyapatite chromatography. In some embodiments, the step of chromatography is mixed mode chromatography. In some embodiments, the step of chromatography is hydroxyapatite chromatography. In some embodiments, the step of chromatography is ion exchange chromatography. In some embodiments, the ion exchange chromatography is anion exchange chromatography. In some embodiments, the ion exchange chromatography is cation exchange chromatography.

In one aspect, provided herein is a method comprising determining the conductivity limit of step of chromatography and adjusting the glycine and/or arginine concentration of the affinity chromatography column elution buffer to the conductivity limit. In some embodiments, the conductivity limit is about ≤30 mS/cm. In some embodiments, the conductivity limit is about 30 mS/cm. In some embodiments, the conductivity limit is about 20 mS/cm. In some embodiments, the conductivity limit is about 10 mS/cm.

In some embodiments, the affinity chromatography column elution buffer has a pH of 3.0-5.0. In some embodiments, the affinity chromatography column elution buffer has a pH of 3.0-3.5, 3.5-4.0, 4.0-4.5 or 4.5-5.0.

In some embodiments, the polypeptide is a recombinant protein. In some embodiments, the polypeptide is an enzyme. In some embodiments, the polypeptide retains specific activity of at least 80% compared to the specific activity before purification. In some embodiments, the polypeptide retains specific activity of at least 85%, 90%, 95%, 96%, 97%, 98%, or 99% compared to the specific activity before purification. In some embodiments, the polypeptide retains specific activity of at least 80%-85%, 85-90%, 90-95%, 95-99% or 100% compared to the specific activity before purification.

In some embodiments, the polypeptide is a recombinant protein. In some embodiments, the polypeptide is an enzyme that retains specific activity of at least 80% compared to the specific activity before purification. In some embodiments, the polypeptide is an enzyme that retains specific activity of at least 85%, 90%, 95%, 96%, 97%, 98%, or 99% compared to the specific activity before purification. In some embodiments, the polypeptide is an enzyme that retains specific activity of at least 80%-85%, 85-90%, 90-95%, 95-99% or 100% compared to the specific activity before purification. In some embodiments, the polypeptide is recombinant human arylsulfatase A. In some embodiments, the polypeptide is recombinant iduronate 2-sulfatase. In some embodiments, the polypeptide is recombinant alpha-galactosidase A. In some embodiments, the polypeptide is a naturally occurring protein. In some embodiments, the polypeptide is a naturally occurring human protein.

In some embodiments, the polypeptide does not comprise an antibody or Fc fusion protein that binds Protein A or Protein G. In some embodiments, the polypeptide does not comprise a domain that binds Protein A or Protein G. In some embodiments, the polypeptide comprises an antigen binding fragment that does not contain an Fc region. In some embodiments, the antigen binding fragment cannot bind Protein A or Protein G. In some embodiments, the Fc region reduces Protein A/G binding, e.g., IgG3 antibodies.

The affinity chromatography elution buffer can be tailored to be compatible with downstream chromatographic resins by including cations (e.g. arginine or salt) or zwitterions (e.g. glycine) and by adjusting the concentrations of glycine and arginine in an elution buffer leading to increased protein yield at a higher, less acidic pH. In some embodiments, adjusting the concentrations of glycine and arginine increases protein activity. In some embodiments, altering the concentrations of glycine and arginine reduces protein aggregation. In some embodiments, altering the concentrations of glycine and arginine increases protein stability.

As described in the Examples section, exemplary protein purification using certain processes described herein conform to the marketing purity requirements in the US and many other countries.

As used in this application, the terms “about” and “approximately” are used as equivalents. Any numerals used in this application with or without about/approximately are meant to cover any normal fluctuations appreciated by one of ordinary skill in the relevant art.

Other features, objects, and advantages of the present invention are apparent in the detailed description that follows. It should be understood, however, that the detailed description, while indicating embodiments of the present invention, is given by way of illustration only, not limitation. Various changes and modifications within the scope of the invention will become apparent to those skilled in the art from the detailed description.

### BRIEF DESCRIPTION OF THE DRAWINGS

The Figures described below, which together make up the Drawings, are for illustration purposes only, not for limitation.

FIG. 1A depicts a chemical equation, showing dissociation of glycine hydrochloride elution buffer. While glycinium cation is the predominant form at low pH, deprotonation leads to the formation of a glycine zwitterion which is the predominant form at mild pH, and has no conductivity. For example, at pH=2.34, half of the glycine exists as a cation, and half exists as a zwitterion. At pH=9.60, half of the glycine exists as anion, and the other half exists as a zwitterion. At pH=5.97, all the glycine exists as zwitterion. So at an acidic pH, for example, in some embodiments, between pH 2.34 and 5.97, glycine is slightly positively charged; while between pH 5.97 and 9.60, glycine is slightly negatively charged. At pH 5.97, glycine has no charge. Within the neutral pH range (between pH 2.34 and 9.60), Glycine generally has low conductivity (majority exists as Zwitterion).

FIG. 1B depicts a conductivity curve for a glycine hydrochloride elution buffer for purifying arylsulfatase A. The conductivity curve depicts a temporary decrease in conductivity at the start of elution.

FIG. 1C is a conductivity curve for a glycine arginine buffer which shows that the decrease in conductivity of a glycine buffer is mitigated by addition of arginine.

FIG. 1D is a graph that shows elution yield of arylsulfatase A in an elution buffer comprising 300 mM Glycine, 30 mM Arginine, pH 3.5. The graph shows that the elution yield was 69%.

FIG. 1E is a graph that shows elution yield of arylsulfatase A in an elution buffer comprising 400 mM Glycine, 10 mM Arginine, pH 3.5. The graph shows that the elution yield was 70%.

FIG. 1F is a graph that shows recombinant arylsulfatase A activity in a 50 mM Glycine-HCl, 50 mM NaCl, pH 3.1 elution buffer after multiple elution cycles as compared to initial protein loaded onto the resin.

FIG. 1G is a graph that shows the percent high molecular weight species (% HMW) in recombinant arylsulfatase A eluted with 50 mM Glycine-HCl, 50 mM NaCl, pH 3.1 elution buffer.

FIG. 2A is a chemical equation that depicts dissociation of acetic acid to yield acetate anion and protons. It is contemplated that as the acetate anion is depleted, dissociation is favored, and proton concentration increases, causing pH instability.

FIG. 2B is an exemplary pH curve, showing that in acetate elution buffer, pH decreased and then gradually increased to elution buffer pH. In contrast, in a glycine-HCl buffer, depletion of chloride anion by the resin has no effect on proton concentration.

FIG. 3A shows elution pH curves for acetate and glycine elution buffer. The acetate buffer generated unstable pH during elution, while glycine buffer did not.

FIG. 3B shows exemplary pH and conductivity curves for two runs, one with 340 mM Glycine-HCl, pH 3.4; and a second run with 142 mM Acetate, pH 3.5.

FIG. 3C shows exemplary conductivity curves for glycine and acetate buffers. A glycine elution buffer showed a temporary conductivity drop, while no conductivity drop was observed when an acetate elution buffer was used.

FIG. 3D shows an exemplary elution curve for arylsulfatase A using a glycine elution buffer.

FIG. 3E shows an exemplary elution curve for arylsulfatase A using an acetate elution buffer.

FIG. 3F is a graph of pH that shows that the elution pool for arylsulfatase A had a pH lower than acetate elution buffer.

FIG. 4 depicts pH and conductivity curves of alpha-galactosidase A purified using affinity chromatography, using an exemplary elution buffer comprising 0.1 M Glycine-HCl, pH 3.0.

FIG. 5 shows a schematic diagram for the procedure of preparing a conductivity vs concentration curve.

FIG. 6 shows a conductivity vs concentration graph, where the concentration upper limit is determined from the plot.

FIG. 7 shows a chromatogram for pH 3.1, pH 3.5, and pH 3.7 glycine elution buffers and depicts the ratio of elution peak area to strip peak area.

FIG. 8 shows an affinity chromatogram using a buffer comprising 300 mM glycine, 50 mM arginine, and conductivity 5.33 mS/cm produced an elution peak larger and narrower than the lower conductivity buffers of 2.87 and 4.26 mS/cm.

FIG. 9A shows a chromatogram at a conductivity of 5.5 mS/cm, where glycine and arginine were combined in different ratios at pH 3.6; and an incFreased ratio of glycine to arginine led to a larger and sharper elution peak, indicating increased product yield.

FIG. 9B shows a chromatogram at a conductivity of between 7.0 and 7.5 mS/cm, glycine and arginine were combined in different ratios at pH 3.6, and increased ratios of glycine to arginine led to larger and sharper elution peaks.

FIG. 9C shows a chromatogram with buffers having comparable conductivity that depicts a wide elution peak and large strip peak with the glycine elution buffer, and where the addition of arginine resulted in a larger and sharper elution peak and a smaller strip peak.

FIG. 10 shows a comparison of affinity chromatograms for acetate and glycine elution buffers.

### DEFINITIONS

In order for the present invention to be more readily understood, certain terms are first defined below. Additional definitions for the following terms and other terms are set forth throughout the specification.

Approximately or about: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Biologically active: As used herein, the phrase “biologically active” refers to a characteristic of any agent that has activity in a biological system, and particularly in an organism. For instance, an agent that, when administered to an organism, has a biological effect on that organism, is considered to be biologically active. In particular embodiments, where a protein or polypeptide is biologically active, a portion of that protein or polypeptide that shares at least one biological activity of the protein or polypeptide is typically referred to as a “biologically active” portion.

Chromatography: As used herein, the term “chromatography” refers to a technique for separation of mixtures. Typically, the mixture is dissolved in a fluid called the “mobile phase,” which carries it through a structure holding another material called the “stationary phase.” Column chromatography is a separation technique in which the stationary bed is within a tube, i.e., column.

Conductivity: As used herein, the term “conductivity” refers to a measure of electrical conduction and shows the ability of an aqueous solution to carry an electrical current.

Diluent: As used herein, the term “diluent” refers to a pharmaceutically acceptable (e.g., safe and non-toxic for administration to a human) diluting substance useful for the preparation of a reconstituted formulation. Exemplary diluents include sterile water, bacteriostatic water for injection (BWFI), a pH buffered solution (e.g. phosphate-buffered saline), sterile saline solution, Ringer's solution or dextrose solution.

Elution: As used herein, the term “elution” refers to the process of extracting one material from another by washing with a solvent. For example, in ion-exchange chromatography, elution is a process to wash loaded resins to remove captured ions. Elution refers to dissociation of binding of target product, e.g. recombinant protein from a resin, and extraction into a solvent or buffer as part of a purification process.

Eluate: As used herein, the term “eluate” refers to a combination of mobile phase “carrier” and the analyte material that emerge from the chromatography, typically as a result of eluting.

Equilibrate or Equilibration: As used herein, the terms “equilibrate” or “equilibration” in relation to chromatography refer to the process of bringing a first liquid (e.g., buffer) into balance with another, generally to achieve a stable and equal distribution of components of the liquid (e.g., buffer). For example, in some embodiments, a chromatographic column may be equilibrated by passing one or more column volumes of a desired liquid (e.g., buffer) through the column.

Improve, increase, or reduce: As used herein, the terms “improve,” “increase” or “reduce,” or grammatical equivalents, indicate values that are relative to a baseline measurement, such as a measurement in the same individual prior to initiation of the treatment described herein, or a measurement in a control individual (or multiple control individuals) in the absence of the treatment described herein. A “control individual” is an individual afflicted with the same form of lysosomal storage disease as the individual being treated, who is about the same age as the individual being treated (to ensure that the stages of the disease in the treated individual and the control individual(s) are comparable).

Impurities: As used herein, the term “impurities” refers to substances inside a confined amount of liquid, gas, or solid, which differ from the chemical composition of the target material or compound. Impurities are also referred to as contaminants.

Load: As used herein, the term “load” refers to, in chromatography, adding a sample-containing liquid or solid to a column. In some embodiments, particular components of the sample loaded onto the column are then captured as the loaded sample passes through the column. In some embodiments, particular components of the sample loaded onto the column are not captured by, or “flow through”, the column as the loaded sample passes through the column.

Peak Splitting: A single protein is typically eluted as a single peak following chromatography. Since different proteins form different peaks, separation between different proteins is achieved. However, if a single protein forms multiple peaks, i.e. peak splitting, it presents as shoulders or twin peaks or distorted peaks with the same baseline. Some factors that contribute to peak splitting are hydrophobicity, loading factor and molecular propensity to aggregation. Peak splitting is undesirable in protein purification as the yield and/or purity are affected. If multiple peaks are collected, purity is reduced, while if a single peak is collected, yield is reduced. It is desirable to minimize peak splitting. In some embodiments, arginine in the elution buffer of the present invention minimizes peak splitting.

Polypeptide: As used herein, a “polypeptide”, generally speaking, is a string of at least two amino acids attached to one another by a peptide bond. In some embodiments, a polypeptide may include at least 3-5 amino acids, each of which is attached to others by way of at least one peptide bond. Those of ordinary skill in the art will appreciate that polypeptides sometimes include “non-natural” amino acids or other entities that nonetheless are capable of integrating into a polypeptide chain, optionally.

Pool: As used herein, the term “pool” in relation to chromatography refers to combining one or more fractions of fluid that has passed through a column together. For example, in some embodiments, one or more fractions which contain a desired component of a sample that has been separated by chromatography (e.g., “peak fractions”) can be “pooled” together generate a single “pooled” fraction.

Soluble: As used herein, the term “soluble” refers to the ability of a therapeutic agent to form a homogenous solution. In some embodiments, the solubility of the therapeutic agent in the solution into which it is administered and by which it is transported to the target site of action is sufficient to permit the delivery of a therapeutically effective amount of the therapeutic agent to the targeted site of action. Several factors can impact the solubility of the therapeutic agents. For example, relevant factors which may impact protein solubility include ionic strength, amino acid sequence and the presence of other co-solubilizing agents or salts (e.g., calcium salts). In some embodiments, therapeutic agents in accordance with the present invention are soluble in its corresponding pharmaceutical composition.

Stability: As used herein, the term “stable” refers to the ability of the therapeutic agent (e.g., a recombinant enzyme) to maintain its therapeutic efficacy (e.g., all or the majority of its intended biological activity and/or physiochemical integrity) over extended periods of time. The stability of a therapeutic agent, and the capability of the pharmaceutical composition to maintain stability of such therapeutic agent, may be assessed over extended periods of time (e.g., for at least 1, 3, 6, 12, 18, 24, 30, 36 months or more). In the context of a formulation, a stable formulation is one in which the therapeutic agent therein essentially retains its physical and/or chemical integrity and biological activity upon storage and during processes (such as freeze/thaw, mechanical mixing and lyophilization). For protein stability, it can be measure by formation of high molecular weight (HMW) aggregates, loss of enzyme activity, generation of peptide fragments and shift of charge profiles.

Viral Processing: As used herein, the term “viral processing” refers to “viral removal,” in which viruses are simply removed from the sample (e.g. viral filtration), or “viral inactivation,” in which the viruses remain in a sample but in a non-infective form. In some embodiments, viral removal may utilize nanofiltration and/or chromatographic techniques, among others. In some embodiments, viral inactivation may utilize solvent inactivation, detergent inactivation, pasteurization, acidic pH inactivation, and/or ultraviolet inactivation, among others.

### DETAILED DESCRIPTION

The present invention provides, among other things, improved methods of purifying a recombinant protein or polypeptide (e.g. including enzymes for enzyme replacement therapy, for example, arylsulfatase A, iduronate 2-sulfatase, alpha-galactosidase A, among others) by affinity chromatography, comprising eluting a polypeptide from an affinity chromatography column using an elution buffer with pH and conductivity properties that are compatible with further downstream purification and processing, thereby minimizing loss of product and product quality. In one aspect, the elution buffer comprises glycine and arginine.

The present invention is based, in part, on the surprising and unexpected finding that affinity resins have an ion exchange effect at the start of elution, causing subsequent conductivity and pH changes on the affinity column, thereby reducing product quality. For example, as the elution buffer contacts the resin, the resin is acidified, and has an anion exchange effect, thereby negatively charged ions bind the resin and are depleted from the solution. The inventors discovered an ion exchange effect in multiple different affinity resins.

Acidic elution buffers commonly used in affinity chromatography cause denaturation of some proteins, leading to reduced yield and activity. Formation of aggregates leading to precipitation also lead to reduced yield. The present invention, provides, in part, affinity elution buffers having compatible pH and conductivity properties with downstream chromatographic steps, and leading to increased protein stability, yield and activity.

In one aspect, provided herein is a method of purifying a polypeptide, comprising loading the polypeptide onto an affinity chromatography column, eluting the polypeptide from the affinity chromatography column using an elution buffer comprising glycine and arginine, subjecting the eluate from the affinity chromatography column to a subsequent chromatography column comprising a resin, wherein conductivity of the elution buffer is no greater than conductivity limit of the resin in the subsequent chromatography column.

In one aspect, provided herein is a method of purifying a polypeptide, comprising loading the polypeptide onto an affinity chromatography column, eluting the polypeptide from the affinity chromatography column using an elution buffer comprising glycine and arginine, wherein conductivity of the elution buffer is no greater than 30 mS/cm. In some embodiments, conductivity of the elution buffer is no greater than 5, 10, 15, 20, 25 or 30 mS/cm. In some embodiments, conductivity of the elution buffer is no greater than 8, 10, 12, 14, 16, 18, 20, 24, 26, 28, 30 mS/cm. In some embodiments, conductivity of the elution buffer is no greater than 10 mS/cm. In some embodiments, conductivity of the elution buffer is about 5-10 mS/cm, 6-10 mS/cm, 7-10 mS/cm, 8-10 mS/cm, or 9-10 mS/cm. In some embodiments, conductivity of the elution buffer is about 5-30 mS/cm, 10-30 mS/cm, 15-30 mS/cm, 20-30 mS/cm, or 25-30 mS/cm.

### Affinity Chromatography

The purification methods described herein can include one or more steps of affinity chromatography (e.g., immuno-affinity chromatography, immobilized metal ion affinity chromatography, and/or immobilized ligand affinity chromatography).

Briefly, affinity chromatography is a chromatographic technique which relies on highly specific interactions, such as, for example, between a receptor and ligand, an antigen and antibody, or an enzyme and substrate. As will be known by the person skilled in the art, selective molecules employed in an affinity chromatography step in the purification methods described herein may be based on various properties (e.g., three dimensional structure, glycosylation, etc.) of recombinant produced protein (e.g., Arylsulfatase A, iduronate 2-sulfatase, alpha-galactosidase A) that can be exploited by the selective molecule. Exemplary selective molecules (or capture reagents) that can be utilized in an affinity chromatography step include a recombinant produced protein, protein A, protein G, an antibody, an antigen binding fragment of an antibody (e.g., VHH), for example, camelid-derived single-domain VHH antibody fragments, a metal ion (e.g., nickel), specific substrate, ligand or antigen. In some embodiments, the affinity capture ligands, e.g., VHH domain affinity ligands are small 14 kD capture ligands that result in high yield and increased purity. In some embodiments, a suitable selective molecule for an affinity chromatography step of the present invention utilizes a recombinant produced protein (e.g., an anti-human Arylsulfatase A antibody). Suitable recombinant produced protein antibodies (e.g., Arylsulfatase A antibodies) or an antigen binding fragment of an antibody may be obtained commercially, through immunization of non-human animals (e.g., a mouse, rat, rabbit, chicken, goat, sheep, horse or other suitable animal for producing antibodies against a human protein), or through in vitro display methods (e.g. bacterial or yeast surface display).

In some embodiments, affinity chromatography resins, including, but not limited to, for example, CaptureSelect®, AVIPure™ or other custom affinity resins are used. For a specific target molecule, affinity ligands (e.g. peptides or proteins) are screened from a library of ligands of different sizes, shapes, conformations and contact areas, and once ligands are identified based on affinity, selectivity, stability and suitable manufacturing properties, affinity ligands are cross-linked onto a resin to generate a custom affinity resin. In some embodiments, the ligand is a small protein molecule (e.g. CaptureSelect®). In some embodiments, the ligand is a small peptide (e.g. AviPure™). In some embodiments, custom affinity resins are selected based on criteria including but not limited to ligand density, chemistry, matrix size, porosity, and linkers.

Generally, a recombinant produced protein (e.g., recombinant Arylsulfatase A) is trapped on a solid or stationary phase or medium through interaction with a selective molecule, while other, undesired molecules are not trapped as they are not bound by the selective molecule(s). The solid medium may then be removed from the mixture, optionally washed, and the molecule of interest released from the entrapment by elution. In some embodiments, affinity columns may be eluted by changing the ionic strength through a gradient. For example, salt concentrations, pH, pI, and ionic strength may be used to separate or to form the gradient to separate.

Provided herein is a method of designing an elution buffer for affinity chromatography with physical (in terms of conductivity, pH) and chemical (in terms of composition of the buffer and dissolved salts) properties to enable compatibility with downstream processes and result in high yield of highly pure and active recombinant protein or polypeptide product.

In some embodiments, a recombinant produced protein may be produced with a tag in order to facilitate purification by affinity chromatography. As will be known by the person skilled in the art, protein tags may include, for example, glutathione-S-transferase (GST), hexahistidine (His), maltose-binding protein (MBP), among others. In some embodiments, lectins are used in affinity chromatography to separate components within the sample. For example, certain lectins specifically bind a particular carbohydrate molecule and can be used to separate glycoproteins from non-glycosylated proteins, or one glycoform from another glycoform.

In some embodiments, elution buffers comprise acetate, citrate, guanidine hydrochloride or glycine hydrochloride. An elution buffer commonly used for affinity purification of proteins is 0.1 M glycine-HCl, pH 2.5-3.0. This buffer effectively dissociates most protein-protein and antibody-antigen binding interactions without permanently affecting protein structure. In some embodiments, amino acids or amino acid derivatives are added to the elution buffer, for example, amino acids including glycine, arginine, proline, lysine, histidine, among others, or amino acid derivatives, for example, arginine derivatives including acetyl-arginine or agmatine. In some embodiments, the elution buffer comprises arginine and glycine. In some embodiments, the elution buffer comprises an arginine derivative and glycine. In some embodiments, the elution buffer comprises a derivative of arginine and a derivative of glycine.

Arginine and glycine are amino acids added to the elution buffer to prevent lowering of conductivity. For example, in an exemplary purification of arylsulfatase A, when a glycine hydrochloride elution buffer is used, negatively charged chloride ions are depleted and the remaining protons and zwitterions have no conductivity. Addition of arginine hydrochloride or sodium chloride prevents the conductivity from dropping too low. Without wishing to be bound by any particular theory, it is contemplated that the resin cannot deplete cations, and the addition of cations to the elution buffer prevents product loss.

Arginine and glycine are added to the elution buffer to prevent lowering of pH. In some embodiments, for example, in purifying arylsulfatase A, when acetate is used as an elution buffer, the acetate anion is depleted by the resin, which causes more acidic radicals, decreasing pH. The resultant pH instability affects product quality. The addition of cations (e.g., arginine) or zwitterions (e.g., glycine) stabilize elution pH and prevent drop in conductivity which benefits product quality.

In one aspect, provided herein is a method of purifying a polypeptide, comprising loading the polypeptide onto an affinity chromatography column, eluting the polypeptide from the affinity chromatography column using an elution buffer comprising glycine and arginine, subjecting the eluate from the affinity chromatography column to a subsequent chromatography column comprising a resin, wherein conductivity of the elution buffer is no greater than conductivity limit of the resin in the subsequent chromatography column.

In one aspect, provided herein is a method of purifying a polypeptide, comprising loading the polypeptide onto an affinity chromatography column, eluting the polypeptide from the affinity chromatography column using an elution buffer comprising glycine and arginine, wherein conductivity of the elution buffer is no greater than 30 mS/cm. In some embodiments, conductivity of the elution buffer is no greater than 5, 10, 15, 20, 25 or 30 mS/cm. In some embodiments, conductivity of the elution buffer is no greater than 10 mS/cm. In some embodiments, conductivity of the elution buffer is no greater than 8, 10, 12, 14, 16, 18, 20, 24, 26, 28, 30 mS/cm. In some embodiments, conductivity of the elution buffer is about 5-10 mS/cm, 6-10 mS/cm, 7-10 mS/cm, 8-10 mS/cm, or 9-10 mS/cm. In some embodiments, conductivity of the elution buffer is about 5-30 mS/cm, 10-30 mS/cm, 15-30 mS/cm, 20-30 mS/cm, or 25-30 mS/cm.

The concentration of glycine in the elution buffer is selected based on conductivity and pH that results in high yield and stability of the protein. For example, in some embodiments, the elution buffer comprises glycine at a concentration no greater than 1500 mM, and/or arginine at a concentration no greater than 300 mM. In some embodiments, the elution buffer comprises glycine at a concentration no greater than 50 mM, 60 mM, 70 mM, 80 mM, 90 mM, 100 mM, 200 mM, 300 mM, 400 mM, 500 mM, 600 mM, 700 mM, 800 mM, 900 mM, 1000 mM, 1100 mM, 1200 mM, 1300 mM, 1400 mM or 1500 mM and/or arginine at a concentration no greater than 100 mM, 150 mM, 200 mM, 250 mM, 300 mM.

In some embodiments, the elution buffer comprises glycine at a concentration of no greater than 500 mM, and/or arginine at a concentration of no greater than 100 mM. In some embodiments, provided herein is a method, wherein the elution buffer comprises glycine at a concentration of no greater than 50 mM, 100 mM, 200 mM, 300 mM, 400 mM or 500 mM, and/or arginine at a concentration of no greater than 10 mM, 20 mM, 30 mM, 40 mM, 50 mM, 60 mM, 70 mM, 80 mM, 90 mM or 100 mM.

In one aspect, provided herein is a method of purifying a polypeptide, the method comprising, loading the polypeptide onto an affinity chromatography column, eluting the polypeptide from the affinity chromatography column using an elution buffer, wherein the elution buffer comprises glycine at a concentration of no greater than 500 mM, and/or arginine at a concentration of no greater than 100 mM.

In one aspect, provided herein is a method of purifying a polypeptide, the method comprising, loading the sample onto an affinity chromatography column, eluting the polypeptide from the affinity chromatography column using an elution buffer, wherein the elution buffer comprises glycine and arginine at a ratio of at least 1:2 (molar ratio). In some embodiments, glycine and arginine are present at a ratio between 1:2 to 40:1 (molar ratio).

In some embodiments, the elution buffer of the method provided herein comprises glycine and arginine at a pH between 3.0-5.0. In some embodiments, the elution buffer of the method provided herein comprises glycine and arginine at a pH between 3.25-4.75. In some embodiments, the elution buffer of the method provided herein comprises glycine and arginine at a pH between 3.0-3.5. In some embodiments, the elution buffer of the method provided herein comprises glycine and arginine at a pH between 3.25-3.75. In some embodiments, the elution buffer of the method provided herein comprises glycine and arginine at a pH between 3.5-4.0. In some embodiments, the elution buffer of the method provided herein comprises glycine and arginine at a pH between 3.75-4.25. In some embodiments, the elution buffer of the method provided herein comprises glycine and arginine at a pH between 4.0-4.5. In some embodiments, the elution buffer of the method provided herein comprises glycine and arginine at a pH between 4.25-4.75. In some embodiments, the elution buffer of the method provided herein comprises glycine and arginine at a pH between 4.5-5.0.

In some embodiments, the elution buffer comprises arginine at a concentration of about 10 mM to 300 mM. In some embodiments, the elution buffer comprises arginine at a concentration of about 10-20 mM, 20-30 mM, 30-40 mM, 40-50 mM, 50-60 mM, 60-70 mM, 70-80 mM, 80-90 mM, 90-100 mM, 100-110 mM, 110-120 mM, 120-130 mM, 130-140 mM, 140-150 mM, 150-160 mM, 160-170 mM, 170-180 mM, 180-190 mM, 190-200 mM, 200-210 mM, 210-220 mM, 220-230 mM, 230-240 mM, 240-250 mM, 250-260 mM, 260-270 mM, 270-280 mM, 280-290 mM, or 290-300 mM.

In some embodiments, the elution buffer comprises glycine at a concentration of about 50 mM to 1500 mM. In some embodiments, the elution buffer comprises glycine at a concentration of about 50-100 mM, 100-150 mM, 150-200 mM, 200-250 mM, 250-300 mM, 300-350 mM, 350-400 mM, 400-450 mM, 450-500 mM, 500-550 mM, 550-600 mM, 600-650 mM, 650-700 mM, 700-750 mM, 750-800 mM, 800-850 mM, 850-900 mM, 900-950 mM, 950-1000 mM, 1000-1100 mM, 1100-1200 mM, 1200-1300 mM, 1300-1400 mM, 1400-1500 mM.

In some embodiments, the method further comprises a step of adjusting the eluate from the affinity chromatography column to a pH of 4.0 to 9.0. In some embodiments, the method further comprises a step of adjusting the eluate from the affinity chromatography column to a pH of 4.0 to 5.0, 5.0-6.0, 6.0-7.0, 7.0-8.0 or 8.0-9.0.

In some embodiments, the elution buffer further comprises a step of viral inactivation of the eluate from the affinity chromatography column.

In some embodiments, the eluate of the affinity column comprises less than 5% high molecular weight aggregates (HMW). In some embodiments, the eluate of the affinity column comprises less than 4% high molecular weight aggregates (HMW). In some embodiments, the eluate of the affinity column comprises less than 3% high molecular weight aggregates (HMW). In some embodiments, the eluate of the affinity column comprises less than 2.5% high molecular weight aggregates (HMW). In some embodiments, the eluate of the affinity column comprises less than 2% high molecular weight aggregates (HMW). In some embodiments, the eluate of the affinity column comprises less than 1% high molecular weight aggregates (HMW).

In some embodiments, the affinity chromatography column uses an antibody or antigen binding fragment of the antibody that specifically binds the polypeptide. In some embodiments, the affinity chromatography column uses variable domains of heavy-chain antibodies that specifically binds the polypeptide. In some embodiments, the affinity chromatography column utilizes antibody-antigen binding interactions to bind polypeptides. For example, in some embodiments, Capture Select affinity chromatography resins bind specific antibody-subdomain regions and are used to specifically target any therapeutic protein, producing high yields of pure therapeutic product. Affinity ligands are designed to have specific affinity, specificity and elution profiles. In some embodiments, mild elution conditions are used to produce stable product.

In some embodiments, the method further comprises adjusting the concentration of arginine and glycine in the elution buffer based on the conductivity limit of the resin for a polypeptide. In some embodiments, the method further comprises subjecting the eluate from the affinity chromatography column to a step of chromatography. In some embodiments, the method comprises subjecting the eluate from the affinity chromatography column to a step of chromatography, wherein the step of chromatography is selected from the group consisting of ion exchange, mixed mode, or hydroxyapatite chromatography. In some embodiments, the step of chromatography is mixed mode chromatography. In some embodiments, the step of chromatography is hydroxyapatite chromatography. In some embodiments, the step of chromatography is ion exchange chromatography. In some embodiments, the ion exchange chromatography is anion exchange chromatography. In some embodiments, the ion exchange chromatography is cation exchange chromatography.

In one aspect, provided herein is a method comprising determining the conductivity limit of step of chromatography and adjusting the glycine and/or arginine concentration of the affinity chromatography column elution buffer to the conductivity limit. In some embodiments, the conductivity limit is about ≤30 mS/cm. In some embodiments, the conductivity limit is about ≤10 mS/cm.

In some embodiments, the affinity chromatography column elution buffer has a pH of 3.0-5.0. In some embodiments, the affinity chromatography column elution buffer has a pH of 3.0-3.5, 3.5-4.0, 4.0-4.5 or 4.5-5.0.

In some embodiments, the polypeptide is a recombinant protein. In some embodiments, the polypeptide is an enzyme. In some embodiments, the polypeptide retains specific activity of at least 80% compared to the specific activity before purification. In some embodiments, the polypeptide retains specific activity of at least 85%, 90%, 95%, 96%, 97%, 98%, or 99% compared to the specific activity before purification. In some embodiments, the polypeptide retains specific activity of at least 80%-85%, 85-90%, 90-95%, 95-99% or 100% compared to the specific activity before purification.

In some embodiments, the polypeptide is a recombinant protein. In some embodiments, the polypeptide is an enzyme that retains specific activity of at least 80% compared to the specific activity before purification. In some embodiments, the polypeptide retains specific activity of at least 85%, 90%, 95%, 96%, 97%, 98%, or 99% compared to the specific activity before purification. In some embodiments, the polypeptide is an enzyme that retains specific activity of at least 80%-85%, 85-90%, 90-95%, 95-99% or 100% compared to the specific activity before purification. In some embodiments, the polypeptide is a lysosomal enzyme, such as glycosidases, proteases and sulfatases. In some embodiments, the polypeptide is recombinant human arylsulfatase A.

In some embodiments, the polypeptide is recombinant iduronate 2-sulfatase. In some embodiments, the polypeptide is recombinant alpha-galactosidase A.

In some embodiments, the polypeptide does not comprise an Fc region that binds Protein A or Protein G. In some embodiments, the polypeptide does not comprise an Fc region.

The affinity chromatography elution buffer can be tailored to be compatible with downstream chromatographic resins by including cations (e.g. arginine or salt) or zwitterions (e.g. glycine) and by adjusting the concentrations of glycine and arginine in an elution buffer leading to increased protein yield at a higher, less acidic pH. In some embodiments, adjusting the concentrations of glycine and arginine increases protein activity. In some embodiments, altering the concentrations of glycine and arginine reduces protein aggregation. In some embodiments, altering the concentrations of glycine and arginine increases protein stability.

In some embodiments, the invention thus provides subjecting the eluate from the affinity chromatography column to one or more subsequent steps of chromatography selected from the group consisting of ion exchange, mixed mode, or hydroxyapatite chromatography

In some embodiments, following affinity chromatography, one or more of the following downstream chromatographic processes are used.

### Ion Exchange Chromatography

The purification methods described herein can include one or more steps of ion exchange chromatography (e.g., anion exchange chromatography and/or cation exchange chromatography).

As is known by a person skilled in the art, ion exchangers (e.g., anion exchangers and/or cation exchangers) may be based on various materials with respect to the matrix as well as to the attached charged groups. For example, the following matrices may be used, in which the materials mentioned may be more or less crosslinked: agarose based (such as SEPHAROSE™ CL-6B, SEPHAROSE™ Fast Flow and SEPHAROSE™ High Performance), cellulose based (such as DEAE SEPHACEL®), dextran based (such as SEPHADEX®), silica based and synthetic polymer based.

The ion exchange resin can be prepared according to known methods. Typically, an equilibration buffer, which allows the resin to bind its counter ions, can be passed through the ion exchange resin prior to loading the sample or composition comprising the polypeptide and one or more contaminants onto the resin. Conveniently, the equilibration buffer can be the same as the loading buffer, but this is not required.

In an optional embodiment of the invention, the ion exchange resin can be regenerated with a regeneration buffer after elution of the polypeptide, such that the column can be re-used. Generally, the salt concentration and/or pH of the regeneration buffer can be such that substantially all contaminants and the polypeptide of interest are eluted from the ion exchange resin. Generally, the regeneration buffer has a very high salt concentration for eluting contaminants and polypeptide from the ion exchange resin.

### Anion Exchange Chromatography

In some embodiments, the recombinant protein or polypeptide is subjected to anion exchange chromatography, e.g., anion exchange chromatography described herein. In some embodiments, the Nuvia Q™ anion exchange filter is used. For the anion exchange resin, the charged groups which are covalently attached to the matrix can be, for example, diethylaminoethyl (DEAE), quaternary aminoethyl (QAE), and/or quaternary ammonium (Q). In some embodiments, the anion exchange resin employed is a Q Sepharose column. The anion exchange chromatography can be performed using, e.g., Q SEPHAROSE™ Fast Flow, Q SEPHAROSE™ High Performance, Q SEPHAROSE™ XL, CAPTO™ Q, DEAE, TOYOPEARL GIGACAP® Q, FRACTOGEL® TMAE (trimethylaminoethyl, a quarternary ammonia resin), ESHMUNO™ Q, NUVIA™ Q, or UNOSPHERE™ Q. Other anion exchangers can be used within the scope of the invention, including but not limited to, but are not limited to, quaternary amine resins or “Q-resins” (e.g., CAPTO™-Q, Q-SEPHAROSE®, QAE SEPHADEX®); diethylaminoethane (DEAE) resins (e.g., DEAE-TRISACRYL®, DEAE SEPHAROSE®, benzoylated naphthoylated DEAE, diethylaminoethyl SEPHACEL®); AMBERJET® resins; AMBERLYST® resins; AMBERLITE® resins (e.g., AMBERLITE® IRA-67, AMBERLITE® strongly basic, AMBERLITE® weakly basic), cholestyramine resin, ProPac® resins (e.g., PROPAC® SAX-10, PROPAC® WAX-10, PROPAC® WCX-10); TSK-GEL® resins (e.g., TSKgel DEAE-NPR; TSKgel DEAE-5 PW); and ACCLAIM® resins.

In embodiments, the anion exchange chromatography is performed using FRACTOGEL® TMAE (trimethylaminoethyl, a quarternary ammonia resin).

In some embodiments, subjecting the recombinant protein or polypeptide sample (e.g. Arylsulfatase A) to the anion exchange chromatography is performed at a temperature about 23° C. or less, about 18° C. or less, or about 16° C. or less, e.g., about 23° C., about 20° C., about 18° C., or about 16° C.

Typical mobile phases for anionic exchange chromatography include relatively polar solutions, such as water, acetonitrile, organic alcohols such as methanol, ethanol, and isopropanol, or solutions containing 2-(N-morpholino)-ethanesulfonic acid (MES). Thus, in certain embodiments, the mobile phase includes about 0%, 1%, 2%, 4%, 6%, 8%, 10%, 12%, 14%, 16%, 18%, 20%, 25%, 30%, 35%, 40%, 45%, 50%, 55%, 60%, 65%, 70%, 75%, 80%, 85%, 90%, 95%, or about 100% polar solution. In certain embodiments, the mobile phase comprises between about 1% to about 100%, about 5% to about 95%, about 10% to about 90%, about 20% to about 80%, about 30% to about 70%, or about 40% to about 60% polar solution at any given time during the course of the separation.

In certain embodiments, for example, a recombinant protein, e.g., rASA is loaded at a binding capacity about 23 AU/L resin or less, e.g., about 19 AU/L resin or less, about 15 AU/L resin or less, or about 12 AU/L resin or less, e.g., between about 12 AU/L resin and about 15 AU/L resin, or between about 15 AU/L resin and about 19 AU/L resin. In some embodiments, the sample of recombinant protein, e.g. arylsulfatase A is loaded onto the anion exchange chromatography column at a binding capacity at least about 4.5 g/L resin (e.g., at least about 5 g/L resin, 6 g/L resin, 7 g/L resin, 8 g/L resin, 9 g/L resin, 10 g/L resin, 11 g/L resin, 12 g/L resin, 13 g/L resin, 14 g/L resin, or 15 g/L resin). In some embodiments, the sample of recombinant protein, e.g. arylsulfatase A is loaded onto the anion exchange chromatography column at a binding capacity ranging between about 4.5-20 g/L resin (e.g., ranging between about 5-20 g/L resin; 5-19 g/L resin, 5-18 g/L resin, 5-17 g/L resin, 5-16 g/L resin, 5-15 g/L resin, 7.5-20 g/L resin, 7.5-19 g/L resin, 7.5-18 g/L resin, 7.5-17 g/L resin, 7.5-16 g/L resin, 7.5-15 g/L resin, 10-20 g/L resin, 10-19 g/L resin, 10-18 g/L resin, 10-17 g/L resin, 10-16 g/L resin, or 10-15 g/L resin).

The aqueous solution comprising the recombinant protein, e.g. Arylsulfatase A and contaminant(s) can be loaded onto the anionic resin as a mobile phase using a loading buffer that has a salt concentration and/or a pH such that the polypeptide and the contaminant bind to the anion exchange resin. The resin can then be washed with one or more column volumes of loading buffer followed by one or more column volumes of wash buffer wherein the salt concentration is increased. Finally, the recombinant protein e.g. Arylsulfatase A can be eluted by an elution buffer of increasing salt concentration. Optionally, elution of the enzyme may also be mediated by gradually or stepwise decreasing the pH. The fractions containing recombinant protein or enzyme (e.g. Arylsulfatase A) activity can be collected and combined for further purification.

In some embodiments, loading the sample of recombinant protein e.g. arylsulfatase A onto the anion exchange chromatography column is performed with a loading buffer. In one embodiment, the loading buffer does not contain sodium chloride. In another embodiment, the loading buffer contains sodium chloride. For example, the sodium chloride concentration of the loading buffer is from about 1 mM to about 25 mM, e.g., from about 1 mM to about 10 mM, from about 1 mM to about 5 mM, or from about 5 mM to about 10 mM. In some embodiments, salt concentration in the mobile phase is a gradient (e.g., linear or non-linear gradient). In some embodiments, salt concentration in the mobile phase is constant. In some embodiments, salt concentration in the mobile phase may increase or decrease stepwise. In some embodiments, loading the sample of recombinant protein, e.g. arylsulfatase A onto the anion exchange chromatography column is performed at a pH from about 5 to about 9, e.g., from about 6 to about 8, e.g., about 7.

In some embodiments, washing the anion exchange chromatography column is performed with one or more washing buffers. For example, washing the anion exchange column can include two or more (e.g., a first and a second) washing steps, each using a different washing buffer. In one embodiment, the washing buffer does not contain sodium chloride. In another embodiment, the washing buffer contains sodium chloride. For example, the sodium chloride concentration of the washing buffer is from about 50 mM to about 200 mM, e.g., from about 50 mM to about 150 mM, from about 100 mM to about 200 mM, or from about 100 mM to about 150 mM, e.g., about 80 mM, about 100 mM, about 120 mM, or about 140 mM. In some embodiments, washing the anion exchange chromatography column is performed at a pH from about 5 to about 9, e.g., from about 6 to about 8, e.g., about 7.

In one embodiment, the elution buffer contains sodium phosphate. For example, the sodium phosphate concentration of the elution buffer is from about 20 mM to about 50 mM, e.g., from about 25 mM to about 45 mM, e.g., about 30 mM, about 35 mM, or about 40 mM. In another embodiment, the elution buffer does not contain sodium chloride. In yet another embodiment, the elution buffer contains sodium chloride. For example, the sodium chloride concentration of the elution buffer is from about 200 mM to about 300 mM, e.g., from about 240 mM to about 280 mM. In some embodiments, eluting the recombinant protein, e.g. arylsulfatase A from the anion exchange chromatography column is performed at a pH from about 5 to about 9, e.g., from about 6 to about 8, e.g., about 7.

In some embodiments, eluting the recombinant protein, e.g. arylsulfatase A from the anion exchange chromatography column includes one or more steps of elution peak collection. For example, the elution peak collection starts from about 50 mAU at the ascending side to about 50 mAU at the descending side, e.g., from about 100 mAU at the ascending side to about 50 mAU at the descending side, from about 200 mAU at the ascending side to about 50 mAU at the descending side, from about 50 mAU at the ascending side to about 100 mAU at the descending side, from about 50 mAU at the ascending side to about 200 mAU at the descending side, or from about 100 mAU at the ascending side to about 100 mAU at the descending side, e.g., as determined by spectrophotometry, e.g., at 280 nM.

It is apparent to the person of ordinary skill in the art that numerous different buffers may be used in the loading, washing, and elution steps. Typically, however, the column can be equilibrated with 1-10 column washes of a buffer comprising 0.05 M MES-Tris, pH 7.0. As of convenience the sample can be loaded in the buffer from the previous step of the purification process, or the sample can be loaded using a loading buffer. The column can be washed with 1-10 column volumes of the buffer used for equilibration, followed by a washing buffer comprising 0.02 MES-Tris, 0.12 M NaCl, pH 7.0. Alternatively, the column can be equilibrated, loaded, and washed with any other equilibration, loading, and washing buffers described herein for anion exchange chromatography. The sample can be eluted in a buffer comprising 0.02 MES-Tris, 0.26 M NaCl, pH 7.0. Alternatively, the sample can be eluted in any other elution buffer described herein for anion exchange chromatography.

The loading buffer, washing buffer, and elution buffer described herein can include one or more buffering agents. For example, the buffering agent can be TRIS, HEPES, MOPS, PIPES, SSC, MES, sodium phosphate, sodium acetate, or a combination thereof. The concentration of the buffering agent is between about 1 mM and about 500 mM, e.g., between about 10 mM and about 250 mM, between about 20 mM and about 100 mM, between about 1 mM and 5 mM, between about 5 mM and 10 mM, between about 10 mM and 50 mM, or between about 50 mM and about 100 mM, e.g., about 1 mM, about 5 mM, about 10 mM, about 20 mM, about 30 mM, about 40 mM, or about 50 mM.

Yield, activity and purity following anion exchange chromatography may vary. In some embodiments, the recombinant protein, e.g. arylsulfatase A activity yield is at least about 75%, e.g., at least about 85%, e.g., between about 85% and about 99%, or between about 90% and about 99%. In some embodiments, the protein yield (AU or Absorbance Units) is from about 10% to 50%, e.g., from about 20% to about 35%, or from about 25% to about 30%, e.g., as determined by spectrophotometry, e.g., at 280 nm. In some embodiments (e.g., those using a TMAE column as described below), the elution pool protein activity yield (AU or Absorbance Units) is from about 70% to 400%, e.g., from about 80% to about 390%, or from about 90% to about 350%, or from about 100% to 150%, greater than at least 95%, e.g., as determined by spectrophotometry, e.g., at 280 nm. In some embodiments (e.g., those using a TMAE column as described below), the host cell protein (HCP) log reduction value (LRV) is between about 0.5 and about 1.1, e.g., between about 0.6 and 0.9, or between about 0.7 and 0.8. In some embodiments (e.g., those using a TMAE column as described below), the purity is at least 75%, e.g., at least 80%, at least 85%, at least 90% or higher, as determined by, for example, capillary electrophoresis-SDS PAGE. In preferred embodiments, the activity yield, HCP LRV and purity (as determined by capillary electrophoresis-SDS PAGE) following anion exchange chromatography are at least about 90%, at least about 0.6 and at least about 80%, respectively.

In preferred embodiments of the invention, an anionic exchange column with a high loading capacity is used. In certain embodiments of the invention, the column is characterized by a loading range between about 3-20 g/L (i.e., about 5-15 g/L, about 10-15 g/L, about 10-20 g/L). In some embodiments, the loading capacity is significantly greater than 4.3 g/L (e.g., is or greater than about 10 g/L, 12.5 g/L, 15 g/L, 17.5 g/L, or 20 g/L). In certain embodiments, the binding capacity of the resin is between about 75-100 AU/L (e.g. about 75 AU/L, about 80 AU/L, about 85 AU/L, about 90 AU/L, about 95 AU/L). In certain embodiments, the loading capacity is greater than about 80 AU/L. In some embodiments, the high load capacity column is a TMAE column. In particular embodiments, the column is selected from the group consisting of a Fractogel® TMAE column, a Nuvia Q column, a Q Sepharose Fast Flow column, a Capto Q column, a Q Sepharose XL column, a Eshmuno Q column, a UNOsphere Q column, or a GigaCap Q column.

In particular embodiments of the invention, a TMAE column is pre-equilibrated with a buffer comprising about 20 mM MES-Tris and 1000 mM NaCl at a pH of 7.0. In certain embodiments, the column is equilibrated with a buffer comprising 50 mM MES-Tris at a pH of 7.0. In some embodiments, the load flow rate of the TMAE column is about 75-125 cm/hr (i.e., about 75-115 cm/hr, about 75-110 cm/hr, about 75-105 cm/hr, about 75-100 cm/hr, about 85-115 cm/hr, about 85-110 cm/hr, about 85-105 cm/hr, about 85-100 cm/hr, about 95-115 cm/hr, about 95-110 cm/hr, about 95-105 cm/hr, about 95-100 cm/hr, about 100-120 cm/hr, about 100-115 cm/hr, about 100-110 cm/hr, about 100 cm/hr). Loading conditions may be optimized and assessed by A280 absorbance as described herein.

In particular embodiments utilizing a TMAE column (e.g., a Fractogel TMAE column), very little product is lost in the flow through during loading, even at loading capacities greater than 15 g/L. The capability of increasing loading capacity while minimizing flow-through loss is a significant improvement in purification methodology. In particular embodiments of the invention, the amount of flow-through product loss is less than 30% of the load (e.g. less than about 25%, less than about 20%, less than about 15%, less than about 10%, or less than about 5%).

After loading, in some embodiments, a TMAE column is washed at least once. In particular embodiments, the column is washed twice. A first or second wash buffer may comprise an optimized level of sodium chloride. In some embodiments, the amount of sodium chloride is a first or second wash buffer is between about 50-150 mM (e.g. about 50-140 mM, about 50-130 mM, about 50-120 mM, about 50-110 mM, about 50-100 mM, about 50-90 mM, about 50-80 mM, about 80-150 mM, about 80-140 mM, about 80-130 mM, about 80-120 mM, about 80-110 mM, about 80-100 mM, about 80-90 mM, about 80 mM, or about 120 mM). In some embodiments, a first wash buffer comprises 50 mM MES-Tris at pH 7.0. In some embodiments, a second wash buffer comprises, 20 mM MES-Tris, 100 mM NaCl at pH 7.0. Further optimization of wash conditions, particularly second wash conditions, is encompassed within embodiments of the present invention. For example, increasing the salt concentration of a second wash may improve host cell protein (HCP) log reduction values (LRV) and overall purity, but decrease both activity and A280 yield. As described herein, particular washing conditions must be balanced with the elution conditions described below in order to provide the optimal combination of purity, activity and yield.

In embodiments of the invention, recombinant ASA bound to a TMAE column is eluted with an elution buffer. In some embodiment, the amount of sodium chloride in the elution buffer is optimized. In particular embodiments, the amount of sodium chloride in the elution buffer is between about 150-300 mM (e.g. about 150-290 mM, about 150-280 mM, about 150-270 mM, about 150-260 mM, about 150-250 mM, about 150-240 mM, about 150-230 mM, about 150-220 mM, about 150-210 mM, about 170-290 mM, about 170-280 mM, about 170-270 mM, about 170-260 mM, about 170-250 mM, about 170-240 mM, about 170-230 mM, about 170-220 mM, about 170-210 mM about 180-290 mM, about 180-280 mM, about 180-270 mM, about 180-260 mM, about 180-250 mM, about 180-240 mM, about 180-230 mM, about 180-220 mM, about 180-210 mM, about 180, about 220 or about 260). In a particular example, the elution buffer comprises 50 mM MES-Tris and 1M NaCl at a pH of 7.0. In some embodiments, the A280 yield following elution is greater than 60% of the load (e.g., about 60%, about 70%, about 80% or higher). Further optimization of elution conditions is encompassed within embodiments of the present invention. For example, increase elution salt concentration (i.e., conductivity) provides better yield but results in poorer purity and HCP removal. And as noted above, particular washing conditions must be balanced with the elution conditions in order to provide the optimal combination of purity, activity and yield.

### Cation Exchange Chromatography

In some embodiments, the method further includes subjecting the sample of recombinant protein, e.g. arylsulfatase A to cation exchange chromatography, e.g., sulfopropyl (SP) cation exchange chromatography, e.g., as described herein. In some embodiments, the sample of recombinant protein, arylsulfatase A is subjected to anion exchange chromatography prior to cation exchange chromatography. In a typical embodiment, the cation exchange chromatography comprises sulfopropyl (SP) cation exchange chromatography, but other cation chromatography membranes or resins can be used, for example, a MUSTANG™ S membrane, an S-SEPHAROSE™ resin, or a Blue SEPHAROSE™ resin. In some embodiments, the method further comprises concentrating and/or filtering the sample of recombinant protein, e.g. arylsulfatase A, e.g., by ultrafiltration and/or diafiltration, e.g., by tangential flow ultrafiltration. The cation exchange chromatography can be performed at an optimized temperature, e.g., as described herein, to enhance target binding and/or decrease impurity binding. For example, the cation exchange chromatography can be performed at a temperature of about 23° C., 18° C., 16° C., or less.

In one embodiment, the cation exchange chromatography includes sulfopropyl (SP) cation exchange chromatography. In another embodiment, the cation exchange chromatography is a polishing step. The cation exchange chromatography (e.g., sulfopropyl (SP) cation exchange chromatography) can be performed using, e.g., one or more of: TOYOPEARL® SP-650, TOYOPEARL® SP-550, TSKGEL® SP-3 PW, TSKGEL® SP-5 PW, SP SEPHAROSE™ Fast Flow, SP SEPHAROSE™ High Performance, SP SEPHAROSE™ XL, SARTOBIND® S membrane, POROS® HS50, UNOSPHERE™ S, and MACROCAP™ S.

The aqueous solution comprising the recombinant protein, e.g. arylsulfatase A and contaminant(s) can be loaded onto the cationic resin using a loading buffer that has a salt concentration and/or a pH such that the polypeptide and the contaminant bind to the cation exchange resin. The resin can then be washed with one or more column volumes of equilibration butter or loading buffer, and optionally followed by one or more column volumes of wash buffer wherein the salt concentration is increased. Finally, the recombinant protein, e.g. arylsulfatase A can be eluted in an elution buffer. The fractions containing recombinant protein, e.g. arylsulfatase A activity can be collected and combined for further purification.

In a typical embodiment, the NaCl concentration and/or pH of the loading buffer, washing buffer, and/or elution buffer, can be optimized, e.g., as described herein, to enhance target binding and/or decrease impurity binding. In some embodiments, the NaCl concentration in the loading buffer is about 20 mM, 15 mM, 10 mM, or less. In some embodiments, the loading buffer has a pH of about 4.5, 4.3, 4.0, or less. In some embodiments, the NaCl concentration in the washing buffer is about 20 mM, 15 mM, 10 mM, or less. In some embodiments, the NaCl concentration in the elution buffer is about 55 mM, 50 mM, 45 mM, 40 mM, or less.

In some embodiments, subjecting the sample of recombinant protein, e.g. arylsulfatase A to a cation exchange chromatography includes: loading the sample of recombinant protein, e.g. arylsulfatase A onto a cation chromatography column (e.g., a sulfopropyl (SP) cation exchange column), washing the cation exchange chromatography column, and eluting the recombinant protein, e.g. arylsulfatase A from the column. In some embodiments, the columns can be equilibrated with more than 3, e.g., 5 to 10 column volumes of 0.01 M NaAc, 0.01 M NaCl, 0.03 M acetic acid, pH 4.2.

In some embodiments, the sample can be loaded in the buffer from the previous step of the purification process, or the sample can be loaded using a loading buffer. In one embodiment, the loading buffer contains sodium chloride. For example, the sodium chloride concentration of the loading buffer is from about 1 mM to about 25 mM, e.g., from about 5 mM to about 20 mM, e.g., about 5 mM, about 10 mM, about 15 mM, or about 20 mM. In another embodiment, the loading buffer contains sodium acetate. For example, the sodium acetate concentration of the loading buffer is from about 10 mM to about 100 mM, e.g., about 20 mM, about 40 mM, or about 60 mM. In some embodiments, loading the sample of recombinant protein, e.g. arylsulfatase A onto the cation exchange chromatography column is performed at a pH from about 3.0 and about 6.0, e.g., from about 4.0 and about 5.0, e.g., about 4.0, about 4.3, or about 4.5. In some embodiments, the sample of recombinant protein, e.g. arylsulfatase A is loaded onto the cation exchange chromatography column at a binding capacity about 15 AU/L resin or less, e.g., about 14 AU/L resin or less, or about 12 AU/L resin or less, e.g., between about 10 AU/L resin and about 14 AU/L resin, or between about 10 AU/L resin and about 12 AU/L resin.

In some embodiments, washing the cation exchange chromatography column is performed with one or more washing buffers. For example, washing the cation exchange column can include two or more (e.g., a first and a second) washing steps, each using a different washing buffer. The column can be washed with 1-10 column volumes of the buffer used for equilibration. Alternatively, the column can be equilibrated, loaded, and washed with any other equilibration, loading, and washing buffers described herein for cation exchange chromatography. In one embodiment, the washing buffer contains sodium chloride. For example, the sodium chloride concentration of the washing buffer is from about 1 mM to about 25 mM, e.g., from about 5 mM to about 20 mM, or from about 10 mM to about 15 mM, e.g., about 5 mM, about 10 mM, about 15 mM, or about 20 mM. In another embodiment, the washing buffer contains sodium acetate. For example, the sodium acetate concentration of the loading buffer is from about 10 mM to about 100 mM, e.g., about 20 mM, about 40 mM, or about 60 mM. In some embodiments, washing the cation exchange chromatography column is performed at a pH from about 3.0 and about 6.0, e.g., from about 4.0 and about 5.0, e.g., about 4.0, about 4.3, or about 4.5.

In some embodiments, eluting the recombinant protein, e.g. arylsulfatase A from the cation exchange chromatography column is performed with an elution buffer. In one embodiment, the elution buffer contains sodium chloride. For example, the sodium chloride concentration of the elution buffer is from about 25 mM to about 75 mM, e.g., from about 45 mM to about 60 mM, e.g., about 45 mM, about 50 mM, about 55 mM, or about 55 mM. In some embodiments, eluting the recombinant protein, e.g. arylsulfatase A from the cation exchange chromatography column is performed at a pH from about 3.0 and about 6.0, e.g., from about 4.0 and about 5.0, e.g., about 4.0, about 4.3, or about 4.5. Thus, as one particular example, the sample can be eluted in a buffer comprising 0.02 M NaAc, 0.05 M NaCl, pH 4.5. Alternatively, the sample can be eluted in any other elution buffer described herein for cation exchange chromatography.

In some embodiments, eluting the recombinant protein, e.g. arylsulfatase A from the cation exchange chromatography column includes one or more steps of elution peak collection. For example, the elution peak collection starts from about 50 mAU at the ascending side to about 50 mAU at the descending side, e.g., from about 100 mAU at the ascending side to about 50 mAU at the descending side, from about 200 mAU at the ascending side to about 50 mAU at the descending side, from about 50 mAU at the ascending side to about 100 mAU at the descending side, from about 50 mAU at the ascending side to about 200 mAU at the descending side, or from about 100 mAU at the ascending side to about 100 mAU at the descending side, e.g., as determined by spectrophotometry, e.g., at 280 nM. Collected eluate peaks may be pooled.

The loading buffer, washing buffer, and elution buffer described herein can include one or more buffering agents. For example, the buffering agent can be TRIS, HEPES, MOPS, PIPES, SSC, MES, sodium phosphate, sodium acetate, or a combination thereof. The concentration of the buffering agent is between about 1 mM and about 500 mM, e.g., between about 10 mM and about 250 mM, between about 20 mM and about 100 mM, between about 1 mM and 5 mM, between about 5 mM and 10 mM, between about 10 mM and 50 mM, or between about 50 mM and about 100 mM, e.g., about 1 mM, about 5 mM, about 10 mM, about 20 mM, about 30 mM, about 40 mM, or about 50 mM.

In some embodiments, subjecting the sample of recombinant protein, e.g. arylsulfatase A to the cation exchange chromatography is performed at a temperature about 23° C. or less, about 18° C. or less, or about 16° C. or less, e.g., about 23° C., about 20° C., about 18° C., or about 16° C. In some embodiments, subjecting the sample of recombinant protein, e.g. arylsulfatase A to the cation exchange chromatography is performed between about 23° C. and about 16° C., e.g., at about 23° C., about 20° C., about 18° C., or about 16° C., and loading the sample of recombinant protein, e.g. arylsulfatase A onto the cation exchange chromatography column is performed at a pH between about 4.5 and about 4.3, e.g., at about 4.5, about 4.4, or about 4.3. In some embodiments, subjecting the sample of recombinant protein, e.g. arylsulfatase A to the cation exchange chromatography is performed at about 23° C. and loading the sample of recombinant protein, e.g. arylsulfatase A onto the cation exchange chromatography column is performed at a pH about 4.5. In some embodiments, subjecting the sample to cation exchange chromatography is performed at about 23° C. and loading the sample of recombinant protein, e.g. arylsulfatase A onto the cation exchange chromatography column is performed at a pH about 4.3. In some embodiments, subjecting the sample of recombinant protein, e.g. arylsulfatase A to the cation exchange chromatography is performed at about 18° C. and loading the sample of recombinant protein, e.g. arylsulfatase A onto the cation exchange chromatography column is performed at a pH about 4.5. In some embodiments, subjecting the sample of arylsulfatase A to the cation exchange chromatography is performed at about 18° C. and loading the sample of arylsulfatase A onto the cation exchange chromatography column is performed at a pH about 4.3.

The yield following cation exchange chromatography may vary. In some embodiments, the recombinant protein, e.g. arylsulfatase A activity yield is at least about 75%, e.g., at least about 80%, e.g., between about 80% and about 105%. In some embodiments, the protein yield (AU or Absorbance Units) is from about 65% to 100%, e.g., from about 70% to about 95%, e.g., as determined by spectrophotometry, e.g., at 280 nm.

The purity and activity following cation exchange chromatography is greatly improved. In some embodiments, the host cell protein (HCP) log reduction value (LRV) is between about 1.0 and about 2.5, e.g., between about 1.5 and about 2.0 or between about 1.7 and about 1.9. In some embodiments, the specific activity of the purified arylsulfatase A is at least from about 50 U/mg to about 140 U/mg, e.g., at least about 70 U/mg, at least about 90 U/mg, at least about 100 U/mg, or at least about 120 U/mg, e.g., as determined by a method described herein. In some embodiments, the recombinant protein, e.g. arylsulfatase A is purified to at least about 95%, at least about 98%, at least about 99%, at least about 99.5%, at least about 99.6%, at least about 99.7%, at least about 99.8%, or at least about 99.9%. The purity of recombinant protein, e.g. arylsulfatase A can be measured by, e.g., one or more of: host cell protein (HCP) Western blot, SDS-PAGE Coomassie staining, SDS-PAGE silver staining, reverse phase HPLC, and size exclusion HPLC. In certain embodiments, decreasing the salt concentration of the loading buffer and lowering its pH enhances binding recombinant proteins, e.g. ASA to the cation exchange column but does not impact impurity binding. In other words, an optimal balance of salt concentration and pH, as set forth above, can increase yield after cation exchange chromatography without adversely affecting purity.

In some embodiments, the pH of a cation exchange eluate pool may be adjusted. In certain embodiments, the pH is adjusted immediately prior to viral filtration. Cation exchange eluate (e.g., SP eluate) may be pH adjusted to about 5.5, about 6.0 about 6.5 or about 7.0 using a pH adjustment buffer comprising 0.25M sodium phosphate, 1.33M sodium chloride, 0.34M sodium citrate, pH 7.0. In certain embodiments, the pH-adjusted SP eluate pool is viral filtered on a Planova 20N filter. In some embodiments, the yield relative to input following viral filtration of pH-adjusted cation exchange eluate is between about 90-100%; i.e., about 90%, about 95%, about 96%, about 97%, about 98%, about 99% or more, as assessed by A280 absorbance. In some embodiments, the yield for viral filtration is significant as it verifies that pH adjustment to about 6.0 allows octamers of ASA (which are about 20 nm in diameter) to dissociate into dimeric form. Thus, the pore size of a viral filter may be selected to ensure that only the dimeric form is filtered (i.e., that the octameric form may be retained by the filter, or cause viral filter plugging). For example, a viral filter with a pore size of 20 nm will retain the octameric form of ASA, but not the dimeric form.

In some embodiments, ion exchange chromatography is followed by mixed mode chromatography.

### Mixed-Mode Chromatography

The purification methods described herein can include one or more steps of mixed-mode chromatography. Mixed-mode chromatography is a type of chromatography in which several modes of separation are applied to resolve a mixture of different molecules, typically in liquid chromatography. For example, a mixed-mode separation can include combinational phases with ion-exchange and reversed phase characteristics at the same time. These stationary phases with more than one interaction type are available from several column manufacturers. In some embodiments, Capto MMC Impres column is used for mixed mode chromatography.

In some embodiments, the method includes subjecting a sample of recombinant protein, e.g. arylsulfatase A to mixed mode chromatography, e.g., mixed mode chromatography described herein, such as a method including ceramic hydroxyapatite (HA) chromatography, e.g., hydroxyapatite type I or type II chromatography. In some embodiments, the mixed mode chromatography is performed using one or more of: CHT™ Ceramic Hydroxyapatite Type I Media, CHT™ Ceramic Hydroxyapatite Type II Media, BIO-GEL® HT Hydroxyapatite, and BIO-GEL® HTP Hydroxyapatite.

In some embodiments, subjecting the sample of recombinant protein, e.g. arylsulfatase A to mixed mode chromatography includes: loading the sample of recombinant protein, e.g. arylsulfatase A onto a mixed mode chromatography column (e.g., HA chromatography), washing the mixed mode chromatography column, and eluting the recombinant protein, e.g. arylsulfatase A from the column. In some embodiments, subjecting the sample of recombinant protein, e.g. arylsulfatase A to the mixed mode exchange chromatography is performed at a temperature about 23° C. or less, about 18° C. or less, or about 16° C. or less, e.g., about 23° C., about 20° C., about 18° C., or about 16° C.

In some embodiments, loading the sample of recombinant protein, e.g. arylsulfatase A onto the mixed mode chromatography column is performed with a loading buffer. In one embodiment, the loading buffer contains sodium phosphate. For example, the sodium phosphate concentration of the loading buffer is from about 1 mM to about 10 mM, e.g., from about 1 mM to about 5 mM, from about 5 mM to about 10 mM, e.g., about 1 mM, about 2 mM, or about 5 mM. In another embodiment, the loading buffer contains sodium chloride. For example, the sodium chloride concentration of the loading buffer is from about 100 mM to about 400 mM, e.g., from about 200 to about 300 mM, e.g., about 220 mM, about 240 mM, about 260 mM, or about 280 mM.

In some embodiments, loading the sample of recombinant protein, e.g. arylsulfatase A onto the mixed mode chromatography column is performed at a pH from about 5 to about 9, e.g., from about 6 to about 8, e.g., about 7.

In some embodiments, the mixed-mode chromatography includes ceramic hydroxyapatite (HA) chromatography. Hydroxyapatite (HAP) usually refers to the crystalline form of calcium phosphate. The mechanism of HAP involves non-specific interactions between negatively charged protein carboxyl groups and positively charged calcium ions on the resin, and positively charged protein amino groups and negatively charged phosphate ions on the resin. Basic or acidic proteins can be adsorbed selectively onto the column by adjusting the buffer's pH; elution can be achieved by varying the buffer's salt concentration. Again, it is evident that numerous buffer compositions as well as combinations of buffers can be employed. Typically, however, the column can be equilibrated with 1-10 column washes of a buffer comprising 0.001 M NaPO₄, 0.02 M MES-Tris, 0.26 M NaCl, pH 7.0. As of convenience the sample can be loaded in the buffer from the previous step of the purification process, or the sample can be loaded using a loading buffer. The column can be washed with 1-10 column volumes of the buffer used for equilibration, followed by a washing buffer comprising 0.005 M NaPO₄, 0.02 M MES-Tris, 0.26 M NaCl, pH 7.0. Alternatively, the column can be equilibrated, loaded, and washed with any other equilibration, loading, and washing buffers described herein for mixed mode chromatography. The sample can be eluted in a buffer comprising 0.04 M NaPO₄, pH 7.0. Optionally, the column can be stripped by washing with 1-10 column volumes of 0.4 M NaPO₄, pH 12. Alternatively, the sample can be eluted in any other elution buffer described herein for mixed mode chromatography.

In some embodiments, washing the mixed mode chromatography column is performed with one or more washing buffers. For example, washing the mixed mode chromatography column can include two or more (e.g., a first and a second) washing steps, each using a different washing buffer.

In one embodiment, the washing buffer contains sodium phosphate. For example, the sodium phosphate concentration of the washing buffer is from about 1 mM to about 10 mM, e.g., from about 1 mM to about 5 mM, from about 5 mM to about 10 mM, e.g., about 1 mM, about 5 mM, or about 10 mM. In another embodiment, the washing buffer contains sodium chloride. For example, the sodium chloride concentration of the washing buffer is from about 50 mM to about 600 mM, e.g., from about 100 mM to about 500 mM, or from about 200 to about 400 mM, e.g., about 220 mM, about 240 mM, about 260 mM, or about 280 mM.

In some embodiments, washing the mixed mode chromatography column is performed at a pH from about 5 to about 9, e.g., from about 6 to about 8, e.g., about 7.

In some embodiments, eluting the recombinant protein, e.g. arylsulfatase A from the mixed mode chromatography column is performed at a pH from about 5 to about 9, e.g., from about 6 to about 8, e.g., about 7. In some embodiments, eluting the arylsulfatase A from the mixed mode chromatography column includes one or more steps of elution peak collection. For example, the elution peak collection starts from about 50 mAU at the ascending side to about 50 mAU at the descending side, e.g., from about 100 mAU at the ascending side to about 50 mAU at the descending side, from about 200 mAU at the ascending side to about 50 mAU at the descending side, from about 50 mAU at the ascending side to about 100 mAU at the descending side, from about 50 mAU at the ascending side to about 200 mAU at the descending side, or from about 100 mAU at the ascending side to about 100 mAU at the descending side, e.g., as determined by spectrophotometry, e.g., at 280 nM.

The loading buffer, washing buffer, and elution buffer described herein can include one or more buffering agents. For example, the buffering agent can be TRIS, HEPES, MOPS, PIPES, SSC, MES, sodium phosphate, sodium acetate, or a combination thereof. The concentration of the buffering agent is between about 1 mM and about 500 mM, e.g., between about 10 mM and about 250 mM, between about 20 mM and about 100 mM, between about 1 mM and 5 mM, between about 5 mM and 10 mM, between about 10 mM and 50 mM, or between about 50 mM and about 100 mM, e.g., about 1 mM, about 5 mM, about 10 mM, about 20 mM, about 30 mM, about 40 mM, or about 50 mM.

In some embodiments, the purification of recombinant protein, e.g. ASA by mixed mode chromatography succeeds the purification by ion-exchange chromatography (e.g., anion exchange chromatography). In some embodiments, it is contemplated, however, that these steps could be performed in the reverse order.

Yield following mixed mode chromatography may vary. In some embodiments, the recombinant protein, e.g. arylsulfatase A activity yield is at least about 80%, e.g., at least about 90%, e.g., between about 80% and about 115%. In some embodiments, the protein yield (AU or Absorbance Units) is from about 30% to 80%, e.g., from about 35% to about 75%, or from about 50% to about 70%, e.g., as determined by spectrophotometry, e.g., at 280 nm.

Purity following mixed mode chromatography is greatly improved. In some embodiments, the specific activity of the purified recombinant protein, e.g. arylsulfatase A is at least from about 50 U/mg to about 140 U/mg, e.g., at least about 70 U/mg, at least about 90 U/mg, at least about 100 U/mg, or at least about 120 U/mg, e.g., as determined by a method described herein. In some embodiments, the arylsulfatase A is purified to at least about 95%, at least about 98%, at least about 99%, at least about 99.5%, at least about 99.6%, at least about 99.7%, at least about 99.8%, or at least about 99.9%. In some embodiments, the purity of arylsulfatase A can be measured by, e.g., one or more of: host cell protein (HCP) Western blot, SDS-PAGE Coomassie staining, SDS-PAGE silver staining, reverse phase HPLC, and size exclusion HPLC. In some embodiments, the host cell protein (HCP) log reduction value (LRV) is between about 0.3 and about 0.6, e.g., between about 0.4 and 0.5.

In some embodiments, mixed mode chromatography is followed by hydrophobic interaction chromatography.

### Hydrophobic Interaction Chromatography (HIC)

The purification methods described herein can include subjecting the sample of recombinant protein, e.g. arylsulfatase A to hydrophobic interaction chromatography (HIC). In one embodiment, the hydrophobic interaction chromatography includes phenyl chromatography. In some embodiments, HIC is carried out using a Capto Phenyl Impres™ column.

In other embodiments, the hydrophobic interaction chromatography includes butyl chromatography or octyl chromatography. In some embodiments, subjecting the sample of arylsulfatase A to HIC is performed at a temperature about 23° C. or less, about 18° C. or less, or about 16° C. or less, e.g., about 23° C., about 20° C., about 18° C., or about 16° C. In some embodiments, the sample of arylsulfatase A is subjected to mixed mode chromatography prior to HIC.

Hydrophobic interaction chromatography utilizes the attraction of a given molecule for a polar or non-polar environment, and in terms of protein, this propensity is governed by the hydrophobicity or hydrophilicity of residues on the exposed, outer surface of a protein. Thus, proteins are fractionated based upon their varying degrees of attraction to a hydrophobic matrix, typically an inert support with alkyl linker arms of 2-18 carbons in chain length. The stationary phase consists of small non-polar groups (butyl, octyl, or phenyl) attached to a hydrophilic polymer backbone (e.g., cross-linked Sepharose™, dextran, or agarose). Thus, the HIC column is typically a butyl SEPHAROSE™ column or a phenyl SEPHAROSE™ column, most typically a phenyl SEPHAROSE™ column.

In some embodiments, the hydrophobic interaction chromatography includes phenyl chromatography using one or more of Phenyl SEPHAROSE™ High Performance, Phenyl SEPHAROSE™ 6 Fast Flow (low sub), or Phenyl SEPHAROSE™ 6 Fast Flow (high sub).

In some embodiments, subjecting the sample of recombinant protein, e.g., arylsulfatase A to hydrophobic interaction chromatography includes: loading the sample of recombinant protein, e.g. arylsulfatase A onto a HIC column, washing the HIC column, and eluting the recombinant protein, e.g. arylsulfatase A from the column. Loading, washing and elution in HIC basically follow the same principle as described above for the ion-exchange chromatography, but often nearly opposite conditions to those used in ion exchange chromatography are applied. Thus, the HIC process involves the use of a high salt loading buffer, which unravels the protein to expose hydrophobic sites. The protein is retained by the hydrophobic ligands on the column, and is exposed to a gradient of buffers containing decreasing salt concentrations. As the salt concentration decreases, the protein returns to its native conformation and eventually elutes from the column. Alternatively proteins may be eluted with PEG.

In some embodiments, loading the sample of recombinant protein, e.g. arylsulfatase A onto the HIC column is performed with a loading buffer. In one embodiment, the loading buffer contains sodium chloride. For example, the sodium chloride concentration of the loading buffer is from about 0.5 M to about 2.5 M, e.g., about 1 M or about 1.5 M. In another embodiment, the loading buffer contains sodium phosphate. For example, the sodium phosphate concentration of the loading buffer is from about 10 mM to about 100 mM, e.g., about 25 mM, about 50 mM, or about 75 mM. In some embodiments, loading the sample of recombinant protein, e.g. arylsulfatase A onto the HIC column is performed at a pH from about 5 to about 7, e.g., from about 5.5 to about 6.5, e.g., about 5.5, about 6.0, or about 6.5. In some embodiments, the sample is loaded onto the HIC column at a binding capacity about 12 AU/L resin or less, e.g., about 10 AU/L resin or less, about 9 AU/L resin or less, about 7 AU/L resin or less, or about 5 AU/L resin or less, e.g., between about 5 AU/L resin and about 9 AU/L resin, or between about 5 AU/L resin and about 7 AU/L resin.

The use of phenyl SEPHAROSE™ as solid phase in the HIC is typical in the present disclosure. Again, it is readily apparent that, when it comes to the exact conditions as well as the buffers and combinations of buffers used for the loading, washing and elution processes, a large number of different possibilities exist. In a typical embodiment, the column can be equilibrated in a buffer which contains 0.05 M NaPO₄, 1 M NaCl, pH 5.5. As of convenience the sample can be loaded in the buffer from the previous step of the purification process, or the sample can be loaded using a loading buffer.

In some embodiments, washing the HIC column is performed with one or more washing buffers. For example, washing the HIC column can include two or more (e.g., a first and a second) washing steps, each using a different washing buffer. In some embodiments, the washing buffer contains sodium chloride. For example, the sodium chloride concentration of the washing buffer is from about 100 mM to about 1.5 M, e.g., from about 250 mM to about 1 M, e.g., about 250 mM, about 500 mM, about 750 mM, or about 1 M. In another embodiment, the washing buffer contains sodium phosphate. For example, the sodium phosphate concentration of the loading buffer is from about 10 mM to about 100 mM, e.g., about 25 mM, about 50 mM, or about 75 mM. In some embodiments, washing the HIC column is performed at a pH from about 5 to about 7, e.g., from about 5.5 to about 6.5, e.g., about 5.5, about 6.0, or about 6.5. For example, washing can be performed using 1-2 column washes of equilibration buffer followed by 1-5 column volumes of 0.02 M MES, 0.05 M NaPO₄, 0.5 M NaCl, pH 5.5. Alternatively, the column can be equilibrated, loaded, and washed with any other equilibration, loading, and washing buffers described herein for HIC.

In some embodiments, eluting the recombinant protein from the HIC column is performed with an elution buffer. In some embodiments, the elution buffer contains sodium chloride. For example, the sodium chloride concentration of the elution buffer is from about 30 mM to about 100 mM, e.g., from about 45 mM to about 85 mM, e.g., about 50 mM, about 60 mM, about 70 mM, or about 80 mM. In some embodiments, eluting the arylsulfatase A from the HIC column is performed at a pH from about 5 to about 9, e.g., from about 6 to about 8, e.g., about 7. For example, in some embodiments, arylsulfatase A can be eluted using 0.02 M MES-Tris, 0.06 M NaCl, pH 7.0. Alternatively, the sample can be eluted in any other elution buffer described herein for HIC.

In some embodiments, eluting the recombinant protein, e.g. arylsulfatase A from the HIC column includes one or more steps of elution peak collection. For example, the elution peak collection starts from about 50 mAU at the ascending side to about 50 mAU at the descending side, e.g., from about 100 mAU at the ascending side to about 50 mAU at the descending side, from about 200 mAU at the ascending side to about 50 mAU at the descending side, from about 50 mAU at the ascending side to about 100 mAU at the descending side, from about 50 mAU at the ascending side to about 200 mAU at the descending side, or from about 100 mAU at the ascending side to about 100 mAU at the descending side, e.g., as determined by spectrophotometry, e.g., at 280 nM.

In some embodiments, the purification of recombinant protein, e.g. arylsulfatase A by HIC succeeds the purification by ion-exchange chromatography (e.g., anion exchange chromatography) and/or mixed mode chromatography. It is contemplated, however, that these steps could be performed in the reverse order.

The loading buffer, washing buffer, and elution buffer described herein can include one or more buffering agents. For example, the buffering agent can be TRIS, HEPES, MOPS, PIPES, SSC, MES, sodium phosphate, sodium acetate, or a combination thereof. The concentration of the buffering agent is between about 1 mM and about 500 mM, e.g., between about 10 mM and about 250 mM, between about 20 mM and about 100 mM, between about 1 mM and 5 mM, between about 5 mM and 10 mM, between about 10 mM and 50 mM, or between about 50 mM and about 100 mM, e.g., about 1 mM, about 5 mM, about 10 mM, about 20 mM, about 30 mM, about 40 mM, or about 50 mM.

Yield following HIC may vary. In some embodiments, the recombinant protein, e.g., arylsulfatase A activity yield is at least about 60%, e.g., at least about 70%, e.g., between about 70% and about 100%. In some embodiments, the protein yield (AU or Absorbance Units) is from about 45% to 100%, e.g., from about 50% to about 95%, or from about 55% to about 90%, e.g., as determined by spectrophotometry, e.g., at 280 nm.

Purity following HIC is greatly improved. In some embodiments, the specific activity of the purified arylsulfatase A is at least from about 50 U/mg to about 140 U/mg, e.g., at least about 70 U/mg, at least about 90 U/mg, at least about 100 U/mg, or at least about 120 U/mg, e.g., as determined by a method described herein.

In some embodiments, the recombinant protein, e.g. arylsulfatase A is purified to at least about 95%, at least about 98%, at least about 99%, at least about 99.5%, at least about 99.6%, at least about 99.7%, at least about 99.8%, or at least about 99.9%. In some embodiments, the purity of arylsulfatase A can be measured by, e.g., one or more of: host cell protein (HCP) Western blot, SDS-PAGE Coomassie staining, SDS-PAGE silver staining, reverse phase HPLC, and size exclusion HPLC. In some embodiments, the host cell protein (HCP) log reduction value (LRV) is between about 0.6 and about 1.2, e.g., between about 0.7 and 0.95.

### Polypeptides and Proteins

It is understood that the present invention is used in the purification of any protein or polypeptide, without limitation, and examples provided herein are merely illustrative. For clarity, based on the mechanism, the present invention applies to any affinity purifications, which rely on protein-protein specific binding. More specifically, this kind of binding relies on amino acid chain interactions. The affinity ligand (immobilized on the stationary phase) needs to be a protein or a peptide, or its derivative. For example, in various embodiments, the proteins or polypeptides to be purified, can include proteins, polypeptides, virus, cell or other particle that has one or more protein components. In some embodiments, the polypeptide or protein is a therapeutic protein. In some embodiments, such therapeutic protein includes antibody-based drugs, anticoagulants, blood factors, bone morphogenetic proteins, engineered protein scaffolds, enzymes, Fc fusion proteins, growth factors, hormones, interferons, interleukins, and thrombolytics. In some embodiments, the polypeptide or protein is an antibody. In some embodiments, the polypeptide or protein is an engineered antibody-based molecule or cell, including IgG monoclonal antibody, FAb fragment, Fc fragment, single chain fragment variable (scFv), heterodimeric IgG-based bispecific antibody, scFv-based bispecific antibody, IgG-scFv-based bispecific antibody, tetravalent scFv-based antibody, IgG-based immunocytokine, tandem scFv-immunocytokine, Fc fusion protein, Fc fusion peptide, antibody drug conjugate, or chimeric antigen receptor (CAR)-T based antibody. In some embodiments, the polypeptide or protein is a monoclonal antibody. In some embodiments, the polypeptide or protein is IgG or its derivative. In some embodiments, the polypeptide or protein is IgA or its derivative. In some embodiments, the polypeptide or protein is IgM or its derivative. In some embodiments, the polypeptide or protein is IgE or its derivative. In some embodiments, the polypeptide or protein is IgD or its derivative. In some embodiments, the polypeptide or protein is a bispecific antibodies. In some embodiments, the polypeptide or protein is a trispecific antibodies. In some embodiments, the polypeptide or protein is antibody-drug conjugates. In some embodiments, the polypeptide or protein is antibody fragments (e.g., scFv, Fab, Fc, F(ab′)₂). In some embodiments, the polypeptide or protein is Fc fusion proteins. In some embodiments, the polypeptide or protein is immunocytokines or its derivative. In some embodiments, the polypeptide or protein is a radioimmunoglobulin or its derivative. The present invention also applies to the purification of a virus, cell or other particles that expresses such protein components on its surface.

In some embodiments, the polypeptide or protein is an enzyme. In some embodiments, the polypeptide or protein is a lysosomal enzyme suitable for enzyme replacement therapy as described in Table 1 below.

TABLE 1
Exemplary Replacement Enzymes for Treatment of Lysosomal Storage Diseases

| Disease | Replacement Enzyme | Substance Stored in disease |
|---|---|---|
| Pompe Disease | Acid- α1,4-Glucosidase | Glycogen α-1-4-linked |
| | | oligosaccharides |
| GM1 Gangliodsidosis | β-Galactosidase | GM₁ Gangliosides |
| Tay-Sachs Disease | β-Hexosaminidase A | GM₂ Ganglioside |
| GM2 Gangliosidosis: AB | GM₂ Activator Protein | GM₂ Ganglioside |
| Variant | | |
| Sandhoff Disease | β-Hexosaminidase A&B | GM₂ Ganglioside |
| Fabry Disease | α-Galactosidase A | Globosides |
| Gaucher Disease | Glucocerebrosidase | Glucosylceramide |
| Metachromatic | Arylsulfatase A | Sulphatides |
| Leukodystrophy | | |
| Krabbe Disease | Galactosylceramidase | Galactocerebroside |
| Niemann Pick, Types A | Acid Sphingomyelinase | Sphingomyelin |
| & B | | |
| Niemann Pick, Type C | Cholesterol Esterification | Sphingomyelin |
| | Defect | |
| Niemann Pick, Type D | Unknown | Sphingomyelin |
| Farber Disease | Acid Ceramidase | Ceramide |
| Wolman Disease | Acid Lipase | Cholesteryl Esters |
| Hurler Syndrome (MPS | α-L-Iduronidase | Heparan and Dermatan |
| IH) | | Sulfates |
| Scheie Syndrome (MPS | α-L-Iduronidase | Heparan and Dermatan |
| IS) | | Sulfates |
| Hurler-Scheie Syndrome | α-L-Iduronidase | Heparan and Dermatan |
| (MPS IS) | | Sulfates |
| Hunter Syndrome (MPS | Iduronate Sulfatase | Heparan and Dermatan |
| II) | | Sulfates |
| Sanfilippo A (MPS IIIA) | Heparan N-Sulfatase | Heparan Sulfate |
| Sanfilippo B (MPS IIIB) | α-N-Acetylglucosaminidase | Heparan Sulfate |
| Sanfilippo C (MPS IIIC) | Acetyl-CoA-Glucosaminide | Heparan Sulfate |
| Sanfilippo D (MPS IIID) | Acetyltransferase N- | Heparan Sulfate |
| | Acetylglucosamine-6- | |
| | Sulfatase | |
| Morquio B (MPS IVB) | β-Galactosidase | Keratan Sulfate |
| Maroteaux-Lamy (MPS | Arylsulfatase B | Dermatan Sulfate |
| VI) | | |
| Sly Syndrome (MPS VII) | β-Glucuronidase | |
| α-Mannosidosis | α-Mannosidase | Mannose/Oligosaccharides |
| β-Mannosidosis | β-Mannosidase | Mannose/Oligosaccharides |
| Fucosidosis | α-L-Fucosidase | Fucosyl Oligosaccharides |
| Aspartyl-glucosaminuria | N-Aspartyl-β- | Aspartylglucosamine |
| | Glucosaminidase | Asparagines |
| Sialidosis (Mucolipidosis | α-Neuraminidase | Sialyloligosaccharides |
| I) | | |
| Galactosialidosis | Lysosomal Protective | Sialyloligosaccharides |
| (Goldberg Syndrome) | Protein Deficiency | |
| Schindler Disease | α-N-Acetyl- | |
| | Galactosaminidase | |
| Mucolipidosis II (I-Cell | N-Acetylglucosamine-1- | Heparan Sulfate |
| Disease) | Phosphotransferase | |
| Mucolipidosis III | Same as ML II | |
| (Pseudo-Hurler | | |
| Polydystrophy) | | |
| Cystinosis | Cystine Transport Protein | Free Cystine |
| Salla Disease | Sialic Acid Transport | Free Sialic Acid and |
| | Protein | Glucuronic Acid |
| Infantile Sialic Acid | Sialic Acid Transport | Free Sialic Acid and |
| Storage Disease | Protein | Glucuronic Acid |
| Infantile Neuronal Ceroid | Palmitoyl-Protein | Lipofuscins |
| Lipofuscinosis | Thioesterase | |
| Mucolipidosis IV | Unknown | Gangliosides & Hyaluronic |
| | | Acid |
| Prosaposin | Saposins A, B, C or D | |

In some embodiments, the polypeptide or protein is a secreted protein. In some embodiments, the polypeptide or protein is a viral protein, for example, for vaccine development. In some embodiments, the polypeptide or protein is a particle that has protein components on its surface. In some embodiments, the polypeptide or protein is a virus or virus-like particle that has protein components on its surface. In some embodiments, the polypeptide or protein is a cell that has protein components on its surface. In some embodiments, the present invention applies to T cells or NK cells, for example, expressing CAR antibodies.

In various embodiments, the present invention applies to any protein or polypeptide which has concerns about high aggregation, low stability, low activity, or low potency, through the elution process. For example, (i) highly engineered antibody based proteins and (ii) recombinant enzymes usually have a complicated structure and biological functions based on its amino acid chain and its glycan(s) structure, and tend to have the concerns of the above.

In some embodiments, the polypeptide or protein is a recombinant protein which comprises at least one active site. In some embodiments, the polypeptide or protein is a recombinant protein which comprises at least two active sites. In some embodiments, the polypeptide or protein is a fusion protein which comprises at least one active site derived from a biologically active protein. In some embodiments, the polypeptide or protein is a fusion protein which comprises at least two active sites derived from a biologically active protein.

In some embodiments, the polypeptide or protein is a glycoprotein. In some embodiments, the polypeptide or protein is a recombinant glycoprotein which comprises at least one glycosylation site. In some embodiments, the polypeptide or protein is recombinant glycoprotein which comprises at least two glycosylation sites. In some embodiments, the polypeptide or protein is recombinant glycoprotein which comprises at least three glycosylation sites. In some embodiments, the polypeptide or protein is a recombinant glycoprotein comprising at least one biologically active site based on amino acids chain. In some embodiments, the polypeptide or protein is a recombinant glycoprotein comprising at least two biologically active sites based on amino acids chain. In some embodiments, the polypeptide or protein is a recombinant glycoprotein comprising at least three biologically active sites based on amino acids chain. In some embodiments, the polypeptide or protein is a recombinant glycoprotein comprising at least one biologically active site based on glycans. In some embodiments, the polypeptide or protein is a recombinant glycoprotein comprising at least two biologically active sites based on glycans. In some embodiments, the polypeptide or protein is a recombinant glycoprotein comprising at least three biologically active sites based on glycans. In some embodiments, the polypeptide or protein is a recombinant glycoprotein comprising at least one biologically active site based on amino acids chain and at least one biologically active site based on glycans. In some embodiments, the polypeptide or protein is a recombinant glycoprotein comprising at least one biologically active site based on amino acids chain and at least two biologically active sites based on glycans. In some embodiments, the polypeptide or protein is a recombinant glycoprotein comprising at least two biologically active sites based on amino acids chain and at least one biologically active site based on glycans. In some embodiments, the polypeptide or protein is a recombinant glycoprotein comprising at least two biologically active sites based on amino acids chain and at least two biologically active sites based on glycans. In some embodiments, the polypeptide or protein is a recombinant glycoprotein comprising engineered glycan structures.

In some embodiments, the polypeptide or protein is a naturally occurring protein. In some embodiments, the polypeptide or protein is a plasma-derived protein, including, for example, but not limited to an immunoglobulin, a coagulation factor, an al-antitrypsin, a fibrin sealant, or an albumin.

Various aspects of the invention are described in further detail in the following examples. The use of examples is illustrative, and not meant to limit the invention.

### EXAMPLES

### Example 1. Effect of Addition of Arginine on Conductivity in Affinity Purification of Recombinant Polypeptides, e.g. Arylsulfatase A using a Glycine Elution Buffer

This example illustrates the effect of addition of arginine in preventing reduced conductivity in an exemplary elution of recombinant polypeptides, e.g. arylsulfatase A purified from an affinity chromatography column using a glycine elution buffer.

Briefly, ASA protein was purified using affinity chromatography, using an exemplary glycine elution buffer. In some exemplary embodiments, the glycine buffer had a concentration of 325 mM Glycine-HCl, pH 3.5.

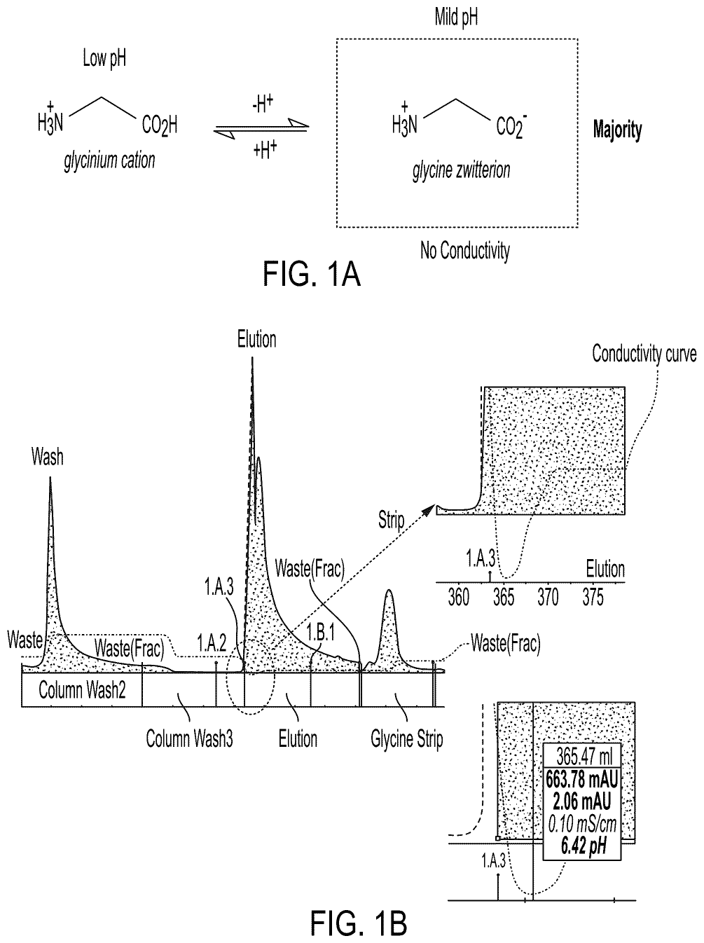

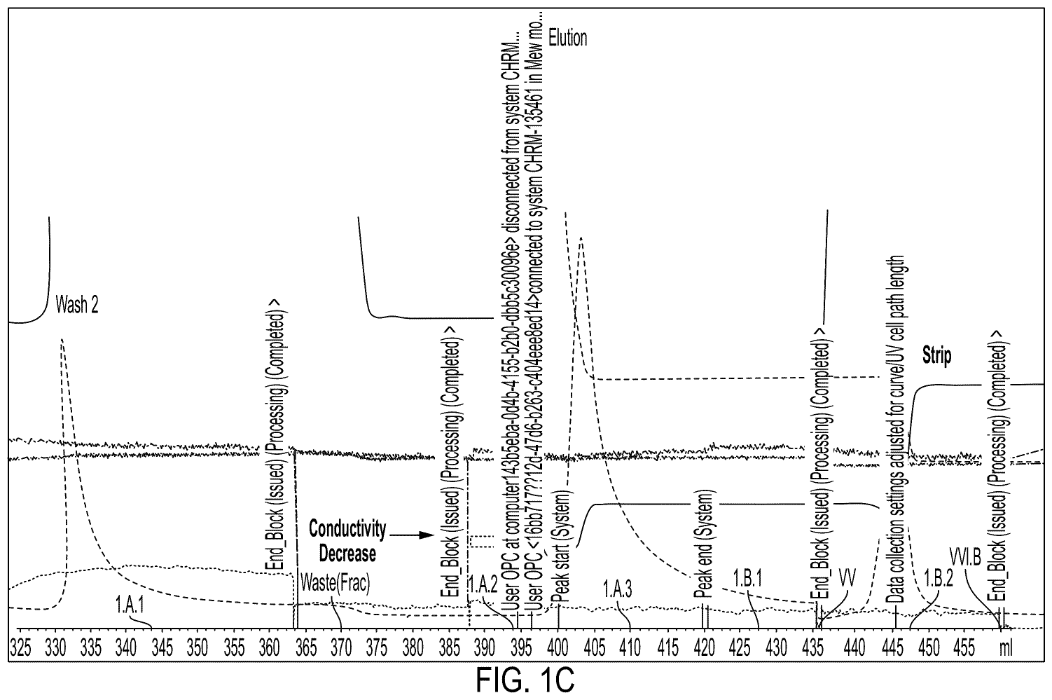

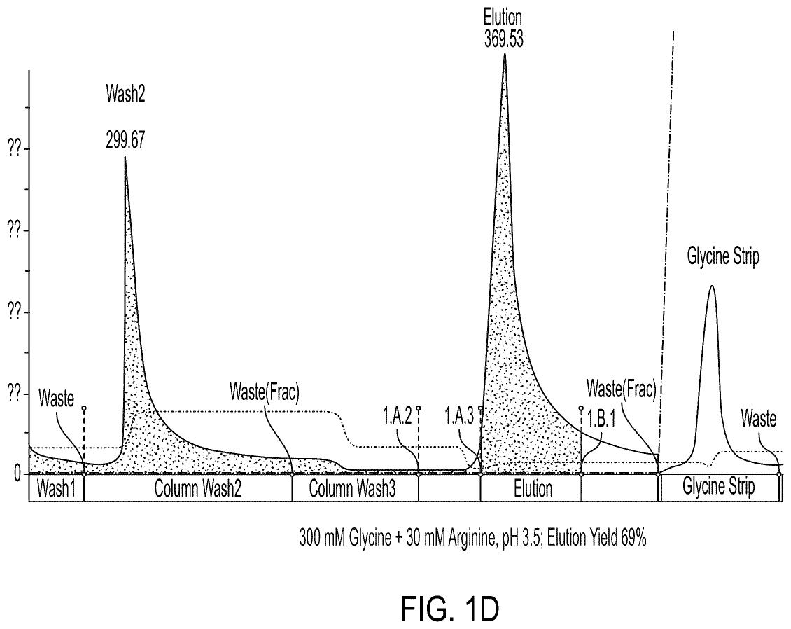

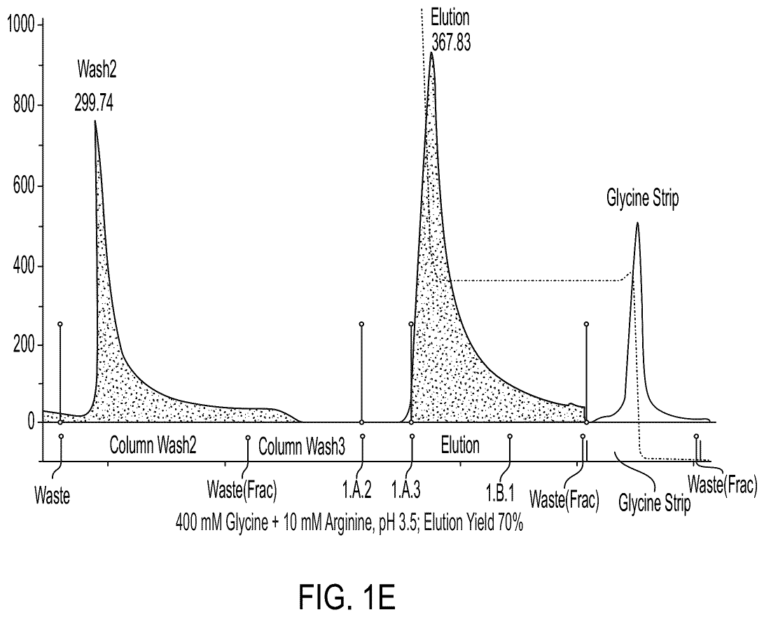

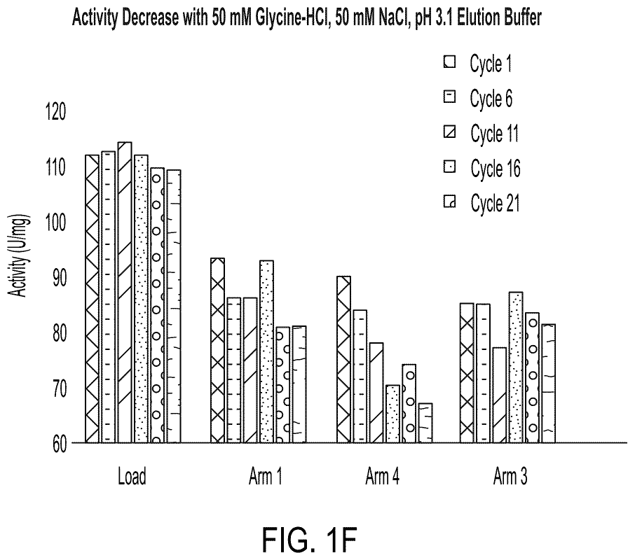

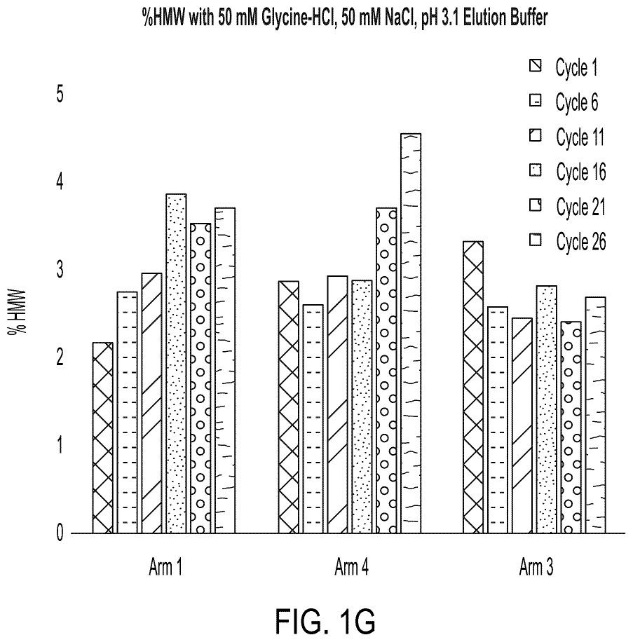

The conductivity was measured during elution using standard methods. As shown in FIG. 1A, at mild pH, the majority of glycine exists as a zwitterion and has no conductivity. The conductivity curve is depicted in FIG. 1B. The results showed that when elution starts, there was a temporary decrease in conductivity (circle with magnified image), almost to zero at the beginning of elution. The conductivity dropped to almost 0 at the beginning of elution, and chloride ions were depleted. Without wishing to be bound by any particular theory, it is contemplated that as the elution buffer contacts the affinity column resin, causing the resin to get acidified and gain anion-exchange function. The chloride ions (Cl⁻) bind with resin, and without chloride, glycine alone in the solution, has about 0 conductivity under neutral pH (pH transition is delayed as compared to conductivity transition). Chloride ions contribute to conductivity, irrespective of pH of the elution buffer, since Cl-ions have no effect on protons.

Arginine was added to the glycine elution buffer, such that the elution buffer comprised 300 mM Glycine-HCl, 30 mM Arginine-HCl, pH 3.5. As shown in FIG. 1C, the conductivity curve following the addition of arginine did not show as much of a decrease in conductivity as compared to the decrease in conductivity in the absence of arginine. Without wishing to be bound by any particular theory, it is contemplated that the addition of arginine provides cations which are not depleted by ion exchange and accordingly, conductivity is better maintained. Combining arginine with glycine also resolved peak splitting.

Overall, the results showed conductivity decreases at the start of elution of arylsulfatase A from an affinity chromatography column with a glycine elution buffer, and that the decrease is mitigated by addition of arginine.

### Example 2. Effect of Addition of Arginine on Yield of Arylsulfatase a in an Affinity Purification Using Glycine Elution Buffer

This example illustrates the effect of addition of arginine in increasing yield in an exemplary elution of arylsulfatase A purified from an affinity chromatography column using a glycine elution buffer.

Briefly, ASA protein was purified using affinity chromatography, using an exemplary glycine elution buffer. In some exemplary embodiments, the glycine buffer had a concentration of 325 mM Glycine-HCl, pH 3.5.

Peak splitting was observed. The elution yield was very low where one peak was collected; for example, at 50 mM Glycine, pH 3.5, the yield was less than 50%. 325 mM Glycine, pH 3.5 a higher elution yield of 70% was observed because both peaks were collected. However, collecting multiple peaks leads to reduced protein purity. (FIG. 1B).

By combining arginine and glycine, peak splitting was no longer observed and instead a single peak of pure protein was observed which was eluted, e.g., 300 mM Glycine in combination with 30 mM Arginine, pH 3.5, the elution yield was 69% (FIG. 1D), and 400 mM Glycine in combination with 10 mM Arginine, pH 3.5; the elution yield was 70% from a single peak (FIG. 1E).

At a constant pH at 3.5, elution yield is optimized by changing glycine to arginine ratio. An exemplary downstream process following affinity chromatography, Nuvia Q Ion Exchange Chromatography has a conductivity limit of 10 mS/cm for the ion exchange resin. In order to achieve conductivity of an arginine-glycine elution buffer below the conductivity limit of the downstream ion exchange resin, an arginine concentration of less than 0.1 M is selected along with a maximum glycine concentration of up to 0.5 M. Various combinations listed in Table 2 are screened to determine the concentration with highest yield.

TABLE 2
Glycine to Arginine Ratio in Elution Buffer

| | | | Arginine + | | | |
|---|---|---|---|---|---|---|
| | Glycine | Arginine | Glycine | Yield | Cycle | |
| Sr. No. | (mM) | (mM) | (mM) | (%) | Number | Resin |
| 1 | 0 | 100 | 100 | 57.7 | 2 | Single lot |
| 2 | 50 | 87.5 | 137.5 | 65.6 | 4 | Single lot |
| 3 | 120 | 75 | 195 | 66.1 | 5 | Single lot |
| 4 | 240 | 50 | 290 | 68.4 | 6 | Single lot |
| 5 | 300 | 30 | 330 | 69 | 17 | Single lot |
| 6 | 300 | 40 | 340 | 81 | 1 | Blend of |
| | | | | | | 3 lots |
| 7 | 325 | 0 | 325 | 70 | 16 | Single lot |
| 8 | 400 | 100 | 410 | 70 | 18 | Single lot |

The results showed that a 100 mM arginine elution buffer resulted in a yield of about 57.7%, while a buffer that comprised 325 mM glycine only had a yield of about 70%. Similarly, 400 mM glycine with 100 mM arginine resulted in a yield of about 70%. Yield was increased to 81% when 40 mM arginine was added to 300 mM glycine.

Overall, results showed that addition of arginine and glycine at concentrations below the conductivity limit of the downstream resin increased yield.

### Example 3. Effect of Addition of Arginine on Arylsulfatase A Protein Peak to an Affinity Chromatography Using Glycine Elution Buffer

This example illustrates the effect of addition of arginine in minimizing peak splitting in an exemplary elution of arylsulfatase A purified from an affinity chromatography column using a glycine elution buffer.

Briefly, ASA protein was purified using affinity chromatography, using an exemplary glycine elution buffer, for example, 50 mM Glycine-HCl, 50 mM NaCl, pH 3.1 elution buffer. The activity of ASA protein was measured after several elution runs of purification on a single column, e.g., cycle 1, 6, 11, 16 and 21.

As shown in FIG. 1F, the baseline activity level is denoted as ‘Load,’ which is a control group, prior to purification cycles. As compared to ‘Load,’ the purified product group (e.g. replicates carried out on different individual columns ARM 1, ARM 4, and ARM 3) showed decreased enzyme activity.

In some embodiments, arginine was added to the glycine elution buffer. In some exemplary embodiments, the glycine buffer had a concentration of 300 mM Glycine-HCl, 30 mM arginine-HCl, pH 3.5 and had a specific activity of 116 U/mg. In some embodiments, the elution buffer had a concentration of 400 mM glycine-HCl and 10 mM arginine-HCl, pH 3.5, and had a specific activity of 116.5 U/mg. Different ARM and different cycles showed variability, however, compared with load, typically, every ARM and cycle showed lower activity than load. On average, ARM product activity was lower than average activity of load. In considering specific cycles, the specific activity of any cycle, e.g. cycle 16, showed a reduction of product activity relative to load. After purification, when eluted with glycine-arginine elution buffer, the protein was found to have similar activity level as the initial load (Table 3, FIG. 1F).

TABLE 3
Specific Activity of Arylsulfatase A

| | | ARM1 | ARM2 | ARM3 |
|---|---|---|---|---|
| | | Product | Product | Product |
| | Activity | Activity | Activity | Activity |
| Load material | (U/mg) | (U/mg) | (U/mg) | (U/mg) |
| Cycle 1 | 112 | 93.1 | 89.8 | 85.1 |
| Cycle 6 | 112 | 86 | 84 | 85 |
| Cycle 11 | 114 | 86.3 | 77.9 | 77.2 |
| Cycle 16 | 111 | 93 | 70.2 | 87.3 |
| Cycle 21 | 109 | 80.5 | 74 | 83.5 |
| Cycle 26 | 109 | 81.1 | 67.2 | 81.4 |
| Average | 111.17 | 86.67 | 77.18 | 82.37 |
| activity | | | | |

Overall, the results showed that addition of arginine to glycine elution buffer resulted in product without any appreciable loss of specific activity, i.e., the specific activity of enzyme in the purified product was comparable to the specific activity of the enzyme in the load, and that addition of arginine to a glycine elution buffer was beneficial to maintaining conductivity levels of arylsulfatase A during elution and preventing or minimizing peak splitting through chromatographic steps, thus a single stable protein was successfully eluted at high yield.

### Example 4. Effect of Addition of Arginine on Arylsulfatase A Protein Aggregation to an Affinity Chromatography Using Glycine Elution Buffer

This example illustrates the effect of addition of arginine in decreasing arylsulfatase A aggregation in an exemplary elution of arylsulfatase A purified from an affinity chromatography column using a glycine elution buffer.

In some embodiments, an affinity chromatography buffer comprising 50 mM Glycine-HCl, 50 mM NaCl, pH 3.1, was used, and the high molecular weight species were quantified as a measure of aggregation (FIG. 1G, Table 4). High aggregation is undesirable as it decreases product yield, activity and quality.

TABLE 4
High Molecular Weight Species (%
HMW) of Arylsulfatase A Product

| Load material | Arm 1 (% HMW) | Arm 4 (% HMW) | Arm 3 (% HMW) |
|---|---|---|---|
| Cycle 1 | 2.16 | 2.83 | 3.29 |
| Cycle 6 | 2.72 | 2.58 | 2.57 |
| Cycle 11 | 2.93 | 2.91 | 2.43 |
| Cycle 16 | 3.82 | 2.84 | 2.79 |
| Cycle 21 | 3.5 | 3.67 | 2.39 |
| Cycle 26 | 3.67 | 4.5 | 2.67 |
| Average % | 3.13 | 3.22 | 2.69 |
| HMW | | | |
| Total Average | | | 3.015 |
| % HMW | | | |

In some embodiments, arginine was added to the glycine elution buffer. For example an elution buffer comprising 300 mM Glycine-HCl, 30 mM Arginine-HCl, pH 3.5 had 2.36% HMW species at cycle 17: In some embodiments, 400 mM Glycine-HCl, 10 mM Arginine-HCl, pH 3.5 had 2.38% HMW at cycle 18.

Overall, the results showed that glycine-arginine buffer reduced the high molecular weight species formed by aggregation and that it had beneficial effects in reducing aggregation.

### Example 5. Comparison between Glycine and Acetate Elution Buffers for Affinity Chromatography

This example illustrates exemplary elution of a recombinant polypeptide, for example, in some embodiments, arylsulfatase A purified from an affinity chromatography column using a acetate elution buffer, and illustrates a comparison between glycine and acetate elution buffers.

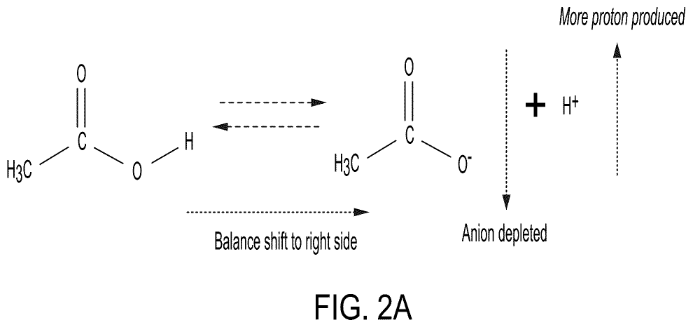

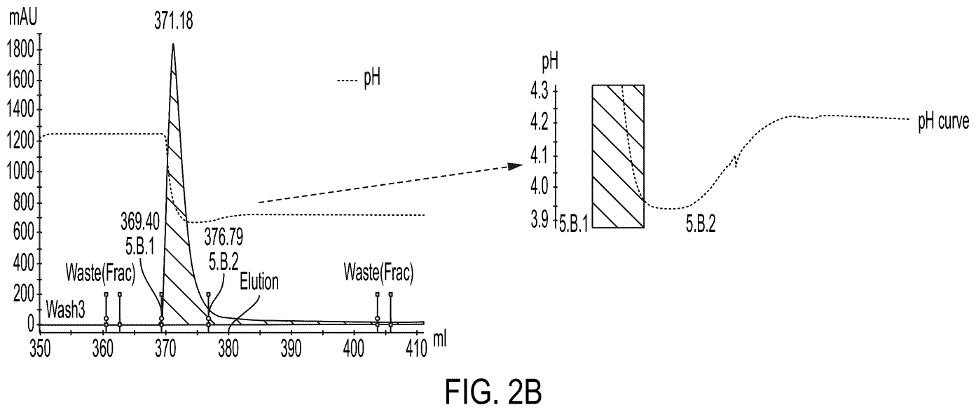

When affinity chromatography elution is carried out using acetate buffer, the CH₃COO⁻ acetate anion is depleted by the resin by anion exchange effect. As shown in FIG. 2A, depletion of acetate anion leads to an increase in H⁺ protons, causing a change in pH. An exemplary acetate elution buffer pH curve is shown in FIG. 2B, showing that the pH decreased and then gradually increased to elution buffer pH. In contrast, in a glycine-HCl buffer, depletion of chloride anion by the resin has no effect on proton concentration.

Briefly, control affinity chromatography runs were carried out with no protein loaded to prevent pH disturbance by protein. An exemplary affinity resin for arylsulfatase A was used and two runs were carried out on the same affinity column, one with 142 mM acetate elution buffer and another with 340 mM glycine elution buffer.

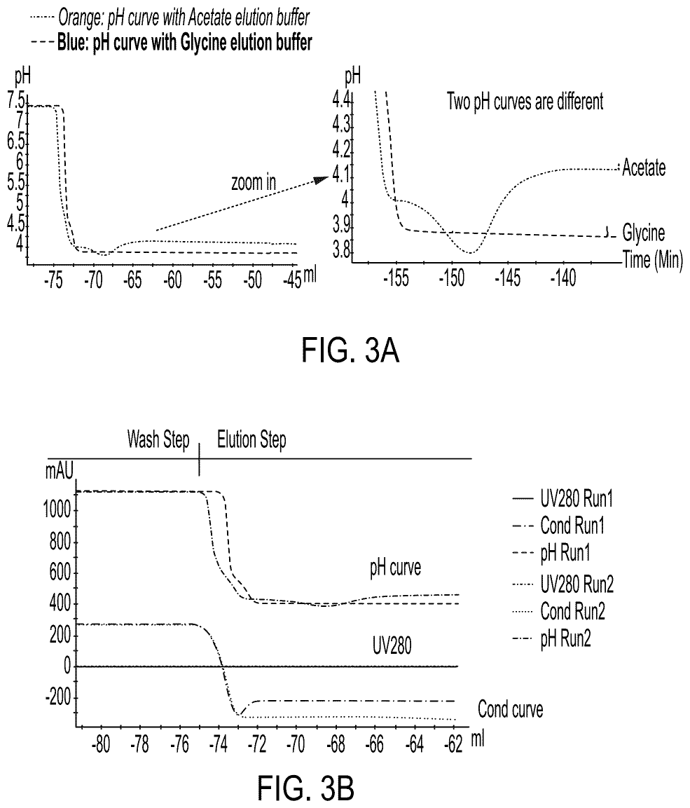

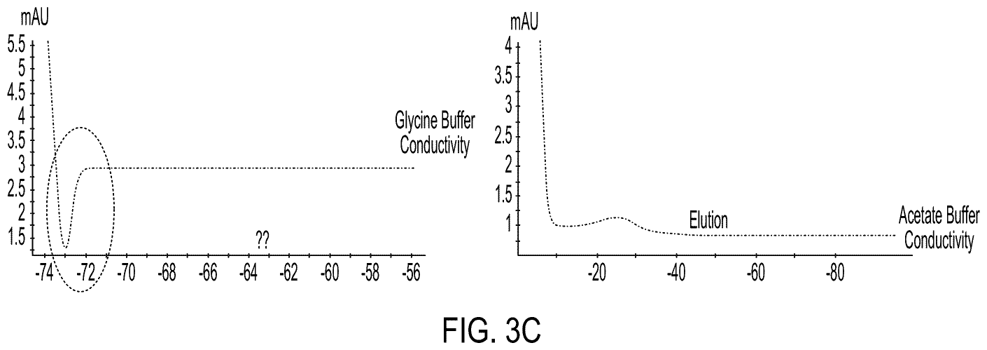

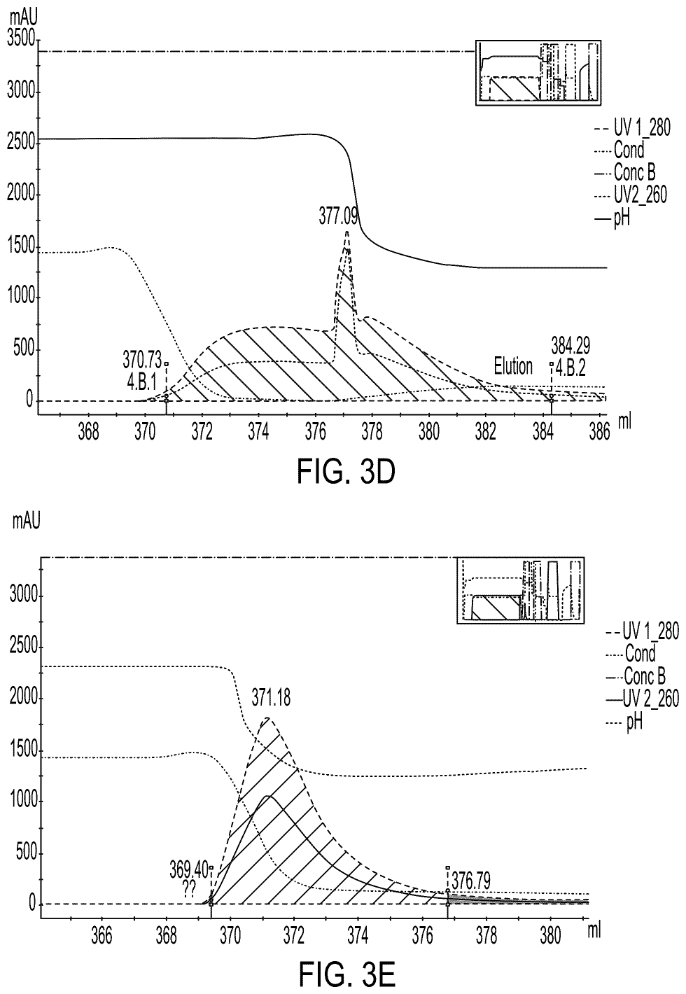

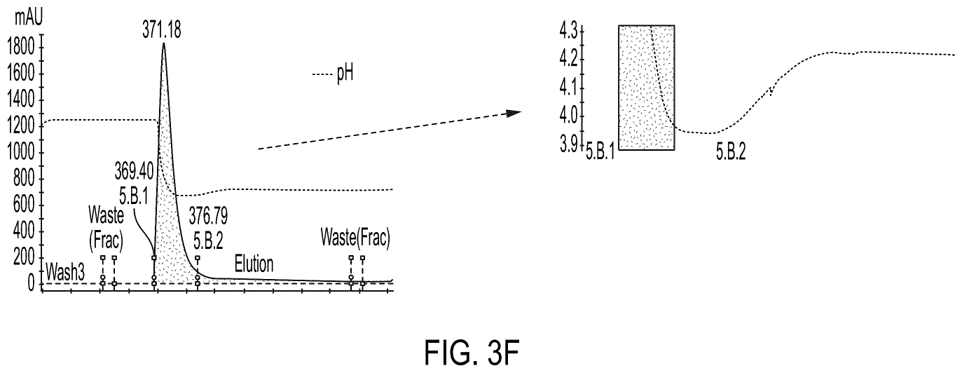

Results shown in FIG. 3A showed a comparison of elution pH curves for acetate and glycine elution buffer. The acetate buffer generated unstable pH during elution. The pH decreased to a low level and then slowly raised back to elution buffer pH. A pH range shift of greater than 0.2 was observed. In some embodiments, the acetate elution pool has a lower pH than acetate elution buffer itself. Without wishing to be bound by any particular theory, it is contemplated that shift in pH generates pockets of low pH on the column which denature protein, leading to reduced product quality.

Exemplary pH and conductivity curves are shown for two runs in FIG. 3B. Run 1 was an exemplary run with 340 mM Glycine-HCl, pH 3.4; Run 2 was carried out with 142 mM Acetate, pH 3.5.

Exemplary conductivity curves using glycine or acetate buffers are shown in FIG. 3C. An elution buffer consisting of glycine only showed a temporary conductivity drop (circle, FIG. 3C). No conductivity drop was observed when an acetate elution buffer was used.

In an exemplary embodiment, elution of arylsulfatase A using a glycine elution buffer (FIG. 3D) and an acetate elution buffer (FIG. 3E) are shown. The results showed that acetate elution buffer generated an earlier pH transition than glycine elution buffer.

With acetate elution buffer, it was found that the elution pool for arylsulfatase A had a pH lower than elution buffer (FIG. 3F). Without wishing to be bound by any particular theory, it is contemplated that this is due to an increase in proton concentration.

Overall, the results from this example showed that pH instability but no conductivity drop occurs when using an acetate elution buffer. In contrast, a glycine elution buffer causes a temporary conductivity drop, but does not cause pH instability.

### Example 6. Anion Exchange Effect in Affinity Purification of Exemplary Recombinant Polypeptide, e.g. alpha-galactosidase A using a Glycine Elution Buffer

This example illustrates that affinity chromatography of exemplary recombinant polypeptides using a glycine elution buffer showed anion exchange effect.

In one embodiment, alpha-galactosidase A protein was purified using affinity chromatography, using an exemplary glycine elution buffer. In some exemplary embodiments, the glycine buffer had a concentration of 0.1 M Glycine-HCl, pH 3.0.

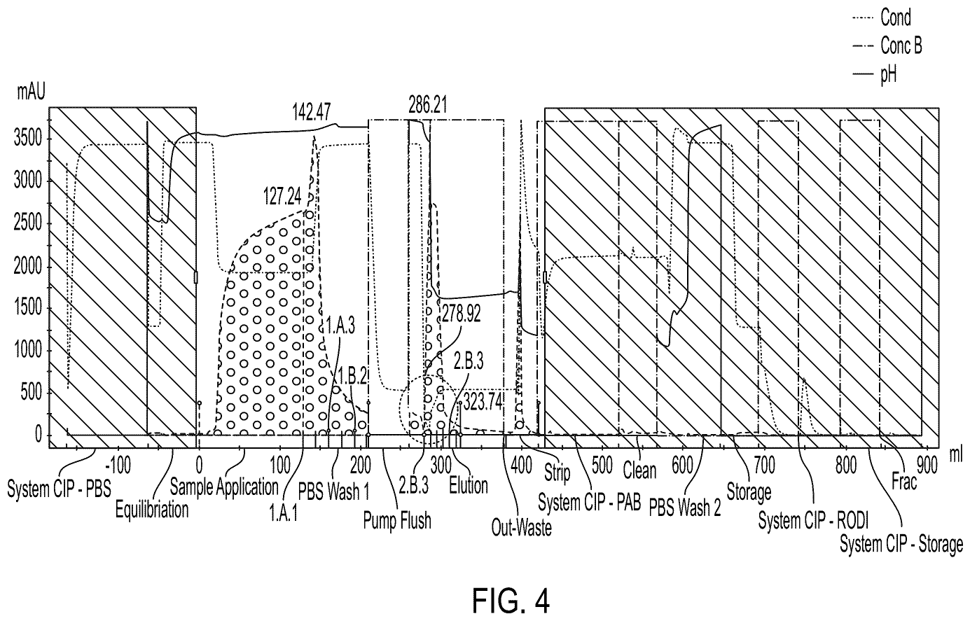

The conductivity was measured during elution using standard methods. As shown in FIG. 4, the results showed that when elution starts, there was a temporary decrease in conductivity to almost 0. Without wishing to be bound by any particular theory, it is contemplated that as the elution buffer contacts the affinity column resin, it causes depletion of chloride ions, and causes the resin to get acidified and gain anion-exchange function.

Overall, the results showed conductivity decreases at the start of elution of alpha-galactosidase A from an affinity chromatography column with a glycine elution buffer.

### Example 7. Determining a Conductivity Limit of Downstream Chromatographic Resin and adjusting amino acid concentration of affinity chromatography elution buffer

This example illustrates an exemplary process of adjusting amino acid concentration of glycine and arginine in an affinity chromatography elution buffer based on conductivity limit of downstream resin.

First, the conductivity limit for the next polishing or purification column after affinity purification is determined. Each resin has a conductivity limit for a specific protein, denoted ‘S’. For example, in an exemplary arylsulfatase A purification process, following affinity purification, the next step uses NuviaQ resin, which has a conductivity limit of about 10 mS/cm as experimentally determined. In an exemplary iduronate 2-sulfatase purification process, following affinity purification, the next step uses hydroxyapatite chromatography, which has a conductivity limit of 30 mS/cm. In an exemplary alpha-galactosidase A purification, the next step after affinity chromatography is ion exchange chromatography, which has a conductivity limit of 10 mS/cm.

Subsequently, the maximum glycine and arginine concentration is used within the conductivity range. The affinity elution buffer pH is determined as X. The loading pH for the next purification column is estimated as Y.

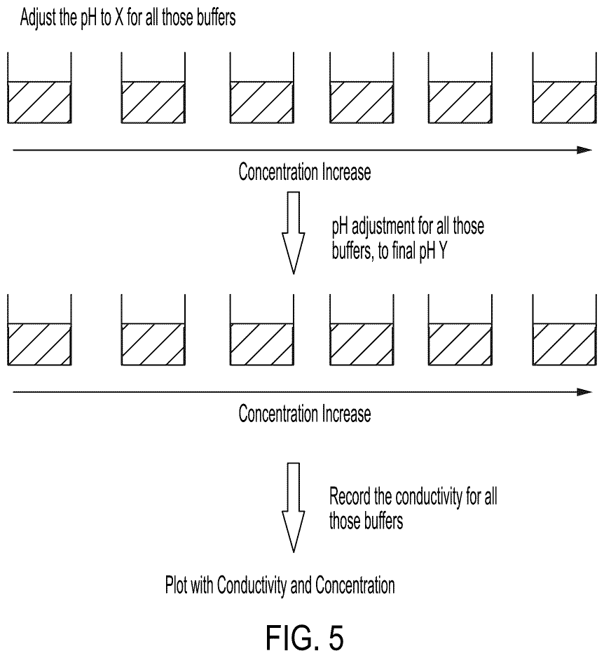

In order to adjust the pH of the elution pool from the affinity purification step to a final pH around Y, a series of glycine solutions with different concentrations are prepared. The pH is adjusted to X with HCl. Then the pH is adjusted to Y, for example with Tris-HCl or Tris base for exemplary arylsulfatase A protein. The final conductivity is measured for all the solutions. The conductivity of the solution is plotted against glycine concentration (FIG. 5).

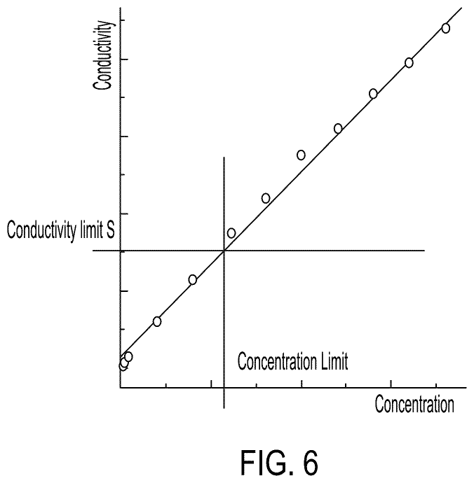

As depicted in FIG. 6, the conductivity vs. concentration curve is a linear curve for both glycine and arginine. Based on the conductivity limit, S, the concentration upper limit for each amino acid is determined from the respective plot. For example, for rhASA, in some embodiments, the glycine maximum limit is 500 mM and the arginine maximum limit is 100 mM. Glycine has very low conductivity after pH is neutralized, while arginine has much higher conductivity.

In some embodiments, for purification of iduronate 2-sulfatase, the maximum glycine limit is around 1500 mM, while the maximum concentration of arginine is around 300 mM. For example, 1 mM glycine conductivity is around 30/1500=0.02 and 1 mM arginine conductivity is about 30/300=0.1. A concentration of glycine and arginine that satisfies the equation 0.02 X+0.1 Y<30 mS/cm is selected, where X mM denotes glycine concentration and Y mM denotes arginine concentration.

In some embodiments, for purification of algasidase alpha, affinity chromatography with 0.1 M glycine, pH 3.0 is followed by ion exchange chromatography, with a conductivity limit of about 10 mS/cm. The maximum concentration of glycine is about 500 mM, while the maximum concentration of arginine is about 100 mM. A concentration of glycine and arginine that satisfies the equation 0.02X+0.1Y<10 is selected, where X mM denotes glycine concentration and Y mM denotes arginine concentration.

Glycine and arginine concentrations are selected so that the total conductivity is not higher than the limit S.

This example illustrates the steps in adjusting the amino acid concentration of an elution buffer for affinity chromatography in an improved process for purifying recombinant protein or polypeptide.

### Example 8. Effect of Addition of Arginine on Conductivity in Affinity Purification of Recombinant Polypeptides, e.g. Iduronate-2-Sulfatase (I2S) Using Glycine Elution Buffer

This example illustrates the effect of addition of arginine in changing conductivity and protein yield in an exemplary elution of recombinant polypeptide, e.g. iduronate-2-sulfatase (I2S), purified from an affinity chromatography column using a glycine elution buffer.

Briefly, I2S protein was purified using affinity chromatography, using exemplary glycine elution buffers. In various embodiments, glycine elution buffers comprising 50 mM glycine-HCl and 50 mM sodium chloride, were titrated using hydrogen chloride to generate elution buffers at various pH ranges, for example, pH 2.9, pH 3.1, pH 3.5, pH 3.7, and pH 4.1. At pH 4.1, 20 mM histidine-HCl was added as a positively charged agent to provide buffering effect.

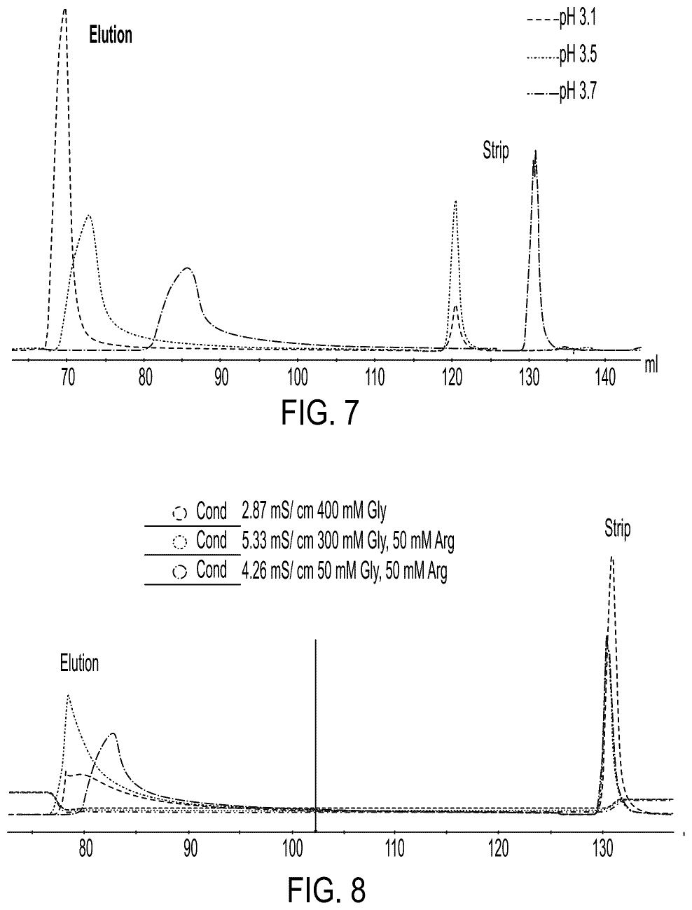

After affinity chromatography with the glycine elution buffer, the estimated percent yield was measured. The results shown in Table 4 demonstrated that increases in pH of the buffer corresponded with decreases in overall yield. Chromatograms for the pH 3.1, pH 3.5, and pH 3.7 glycine elution buffers revealed flatter elution peaks and larger strip peaks indicating lower product yield. Higher ratio of elution peak area to strip peak area indicated higher yield, while a narrower and sharper elution peak was more convenient for collection (FIG. 7).

TABLE 5
Estimated Percent Yield of Iduronate-2-Sulfatase Product

| Elution pH (50 mM glycine, 50 | Estimated Percent |
|---|---|
| mM NaCl) | Yield |
| 2.9 | 106% |
| 3.1 | ~100% |
| 3.5 | 82% |
| 3.7 | 75.6% |
| 4.1 | 51.7% |

The specific activity of I2S was measured after affinity chromatography with various elution buffers. In some embodiments, arginine was added to the glycine elution buffer, such that the elution buffer comprised 50 mM glycine-HCl, 50 mM arginine-HCl, pH 3.6. As shown in Table 6, the combination of glycine and arginine had no significant impact on I2S specific activity at about pH 3.6.

TABLE 6
Specific Activity of Iduronate-2-Sulfatase.

| Sample Name | Specific Activity (U/mg) |
|---|---|
| Assay Control (DPS11022) | 4.03 |
| 50 mM Gly, 50 mM Arg, 20 mM His, pH 4.1 | 3.8 |
| 50 mM Gly. 50 mM Arg, pH 3.6 | 3.52 |
| 100 mM Arg, pH 3.6 | 3.46 |
| 50 mM Gly, 50 mM NaCl pH 3.7 | 3.79 |

At an exemplary constant pH 3.6, elution yield was optimized by changing the glycine to arginine ratio. To allow for multiple downstream processing options, the conductivity limit of, iduronate-2-sulfatase was 10 mS/cm. As such, the maximum glycine and arginine concentrations used were within this conductivity range at pH 3.6.

As depicted in FIG. 8, the conductivity influenced the elution and strip peak on the affinity chromatogram. In this example, the buffer comprising 300 mM glycine, 50 mM arginine, conductivity 5.33 mS/cm produced an elution peak larger and narrower than the lower conductivity buffers of 2.87 and 4.26 mS/cm, indicating a higher protein yield and in some embodiments, also higher product quality.

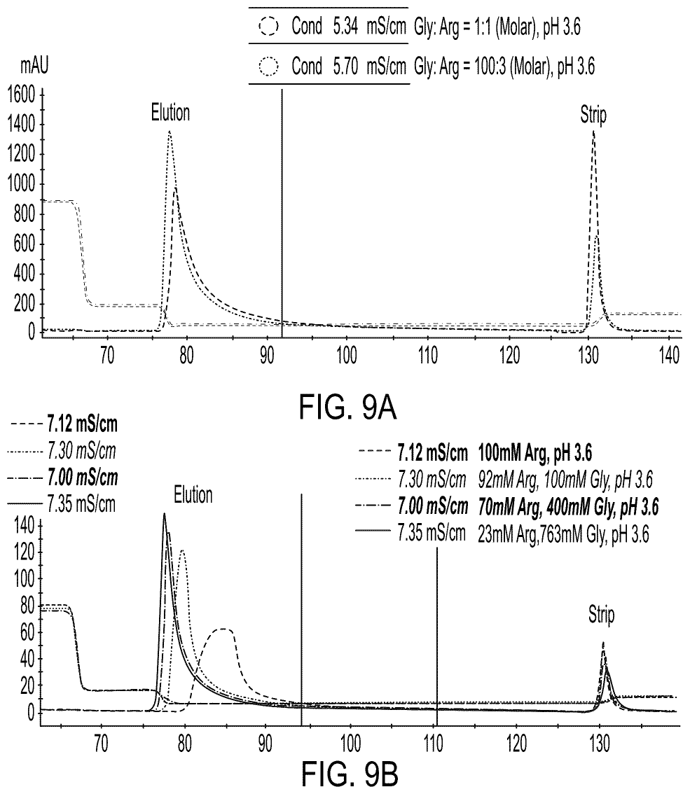

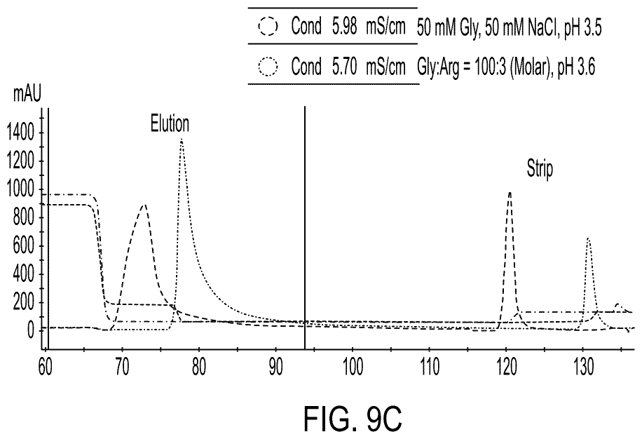

At an exemplary conductivity around 5.5 mS/cm, glycine and arginine were combined in different ratios at pH 3.6, and the effect on product yield was measured. As shown in FIG. 9A, increased ratio of glycine to arginine led to a larger and sharper elution peak, indicating increased product yield.

At exemplary conductivity of between 7.0 and 7.5 mS/cm, glycine and arginine were again combined in different ratios at pH 3.6. As shown in FIG. 9B, increased ratios of glycine to arginine led to larger and sharper elution peaks.

Overall, the results showed that addition of arginine to glycine at higher glycine to arginine ratios below the conductivity limit of downstream processes increased protein yield.

### Example 9. Effect of Addition of Arginine on Yield of Iduronate-2-Sulfatase in an Affinity Purification Using Glycine Elution Buffer

This example illustrates the effect of addition of arginine in increasing yield in an exemplary elution of iduronate-2-sulfatase purified from an affinity chromatography column using a glycine elution buffer.

Briefly, I2S protein was purified using affinity chromatography, using an exemplary glycine elution buffer. In some embodiments, sodium chloride was added to the glycine buffer such that the buffer comprised 50 mM glycine, 50 mM sodium chloride, pH 3.5, conductivity 5.98 mS/cm.

In some embodiments, arginine was then added to an exemplary glycine buffer at a 100:3 glycine to arginine ratio and a similar conductivity such that the glycine elution buffer comprised 606.4 mM glycine, 18.2 mM arginine, pH 3.6, conductivity 5.70 mS/cm.

The results in FIG. 9C showed that a wide elution peak and large strip peak were observed with the glycine elution buffer. The addition of arginine resulted in a larger and sharper elution peak and a smaller strip peak, indicating enhanced product yield. In FIG. 9C, the two buffers tested had almost the same conductivity. Under the same conductivity, usually the lower pH (harsher pH) will generate higher elution yield. By using glycine and arginine combination, we could achieve higher yield under a higher pH (milder pH), which is unusual. Comparing with pH 3.5, pH 3.6 was a milder condition and potentially could improve the product quality. We still could achieve higher yield even under a milder elution condition. This indicated that we could improve both the yield and product quality based on this approach.

Overall, the results showed that addition of arginine to a glycine elution buffer increased the product yield, even at a similar overall conductivity. The results also showed that increasing the glycine concentration, even without changing the overall pH and conductivity could increase the product yield.

### Example 10. Effect of Addition of Arginine on Specific Activity of Iduronate-2-Sulfatase in an Affinity Purification Using Glycine Elution Buffer

This example illustrates the effect of addition of arginine on specific activity in an exemplary elution of iduronate-2-sulfatase purified from an affinity chromatography column using a glycine elution buffer.

Briefly, I2S protein was purified using affinity chromatography, using an exemplary glycine elution buffer, for example 1000 mM glycine, 30 mM arginine, pH 3.6. The specific activity of purified I2S protein was measured after several elution runs with different elution buffers.

As shown in Table 7, the baseline activity level was denoted as ‘Assay Control,’ which is a control group using standard elution buffer. As compared to ‘Assay Control,’ the purified product with glycine elution buffers showed no decrease in enzyme activity.

TABLE 7
Specific Activity of Iduronate-2-Sulfatase

| Sample Name | Specific Activity (U/mg) |
|---|---|
| 50 mM sodium acetate, 1.0M sodium chloride, | 2.73 |
| 20% propylene glycol, pH 4.5 (Standard) | |
| 1000 mM Glycine, 30 mM Arginine, pH 3.6 | 3.84 |
| 100 mM Glycine, 92 mM Arginine, pH 3.6 | 2.84 |
| 400 mM Glycine, 70 mM Arginine, pH 3.6 | 3.75 |
| 50 mM Glycine, 50 mM NaCl, pH 3.1 | 2.97 |
| 100 mM Arginine, pH 3.6 | 2.91 |

In some embodiments, arginine was added to the glycine elution buffer. In some exemplary embodiments, the glycine buffer had a concentration of 1000 mM glycine-HCl, 30 mM arginine-HCl, pH 3.6 and had a specific activity of 3.84 U/mg. In some embodiments, the elution buffer comprised 50 mM glycine-HCl, 50 mM NaCl, pH 3.1 and had a specific activity of 2.91 U/mg. Different buffers showed different specific activities; however, compared to ‘Assay Control,’ all tested buffers showed comparable if not increased specific activity.

Overall, the results showed that a glycine elution buffer with the addition of arginine provided comparable if not increased product quality, i.e., the specific activity of enzyme in the purified product with an arginine supplemented glycine buffer was comparable or increased compared to the specific activity of the enzyme in the previously standard buffer.

### Example 11. Effect of Addition of Arginine on Iduronate-2-Sulfatase Protein Aggregation to an Affinity Chromatography Using Glycine Elution Buffer

This example illustrates the addition of arginine in decreasing iduronate-2-sulfatase aggregation in an exemplary elution of iduronate-2-sulfatase from an affinity chromatography column using a glycine elution buffer.

Briefly, the aggregation of purified product was measured, in this example, by measuring the percentage of high molecular weight species after purification by affinity chromatography using various elution buffers. High aggregation is undesirable as it represents poor product quality.

The results depicted in Table 7 indicated that 50 mM glycine with 50 mM sodium chloride elution buffer at pH 3.1 contained 7.74% high molecular weight species. With 1000 mM glycine and 30 mM arginine buffer at pH 3.6, the high molecular weight species were 0.29% of total product.

TABLE 8
Specific Activity of Iduronate-2-Sulfatase

| | Aggre- | Aggre- | |
|---|---|---|---|
| | gate | gate | HMW % |
| Elution Buffers | A % | B % | Total |
| 1000 mM Glycine, 30 mM Arginine, pH | 0.02 | 0.27 | 0.29 |
| 3.6 | | | |
| 100 mM Glycine, 92 mM Arg, pH 3.6 | 0.37 | 0.83 | 1.2 |
| 400 mM Glycine, 70 mM Arginine, pH | 0.39 | 0.71 | 1.1 |
| 3.6 | | | |
| 50 mM Glycine, 50 mM NaCl, pH 3.1 | 0 | 7.74 | 7.74 |
| 100 mM Arginine, pH 3.6 | 0.58 | 1.03 | 1.61 |
| 50 mM Sodium Acetate 1.0M NaCl 20% | 0.24 | 0.68 | 0.92 |
| Propylene Glycol, pH 4.5 | | | |
| 50 mM Glycine, 50 mM Arginine, 20 mM | 0.19 | 0.68 | 0.87 |
| Histidine, pH 4.1 | | | |
| 50 mM Glycine, 50 mM Arginine, pH 3.6 | 0.74 | 1.01 | 1.75 |
| 300 mM Glycine, 50 mM Arginine, pH | 0.32 | 0.69 | 1.01 |
| 3.6 | | | |
| 50 mM Glycine, 50 mM NaCl, pH 3.7 | 0.37 | 0.85 | 1.22 |

Overall, the results showed that addition of arginine at high glycine to arginine ratios reduced the high molecular weight species formed by aggregation and that it had beneficial effects in reducing aggregation.

### Example 12. Comparison Between Glycine and Acetate Elution Buffers for Iduronate-2-Sulfatase Affinity Chromatography

This example illustrates exemplary elution of a recombinant polypeptide, iduronate-2-sulfatase purified from an affinity chromatography column using an acetate elution buffer and illustrates a comparison between acetate and glycine elution buffers.

Briefly, affinity chromatography runs were carried out with iduronate-2-sulfatase. An acetate elution buffer, comprising 50 mM sodium acetate, 1.0 M sodium chloride, 20% propylene glycol, pH 4.5, was compared to a glycine elution buffer, comprising 1 M glycine, 30 mM arginine, pH 3.6.

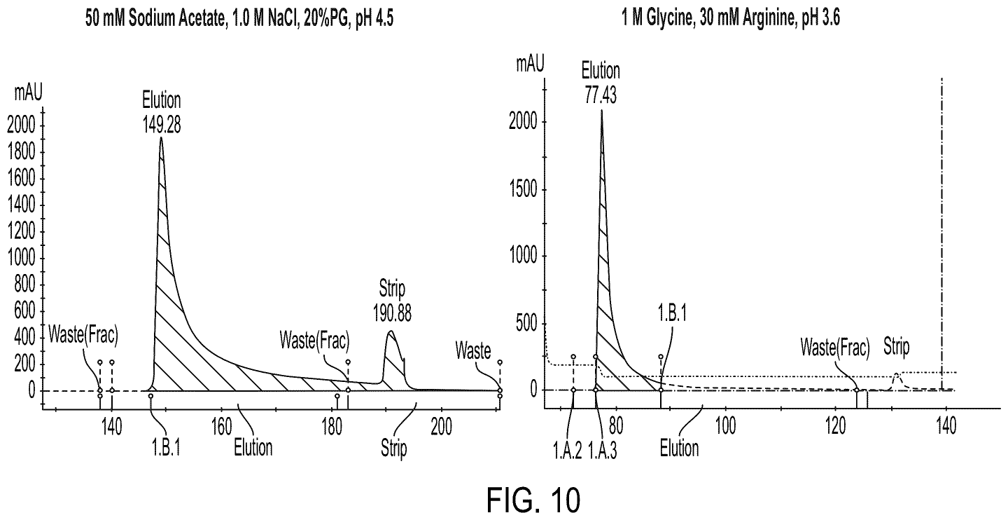

Results in FIG. 10 showed a comparison of affinity chromatograms for acetate and glycine elution buffers. Similar yields of around 90% were recorded for each run. However, the glycine elution peak was sharper than the acetate elution peak denoting a higher concentration of protein in the product and lower pool volume for collection.

Additionally, the conductivity of each buffer was measured. The acetate buffer had a conductivity of 51.0 mS/cm while the glycine buffer had a conductivity of 9.4 mS/cm, which would allow different downstream processes.

Overall, the results from this example showed that the glycine elution buffer provided both a higher protein concentration and lower conductivity, providing conditions that show enhanced favorability for downstream operations.

1. A method of purifying a polypeptide, comprising
loading the polypeptide onto an affinity chromatography column,
eluting the polypeptide from the affinity chromatography column using an elution buffer comprising glycine and arginine,
subjecting the eluate from the affinity chromatography column to a subsequent chromatography column comprising a resin,
wherein conductivity of the elution buffer is no greater than conductivity limit of the resin in the subsequent chromatography column, and wherein the conductivity of the elution buffer is no greater than 30 mS/cm.

2-3. (canceled)

4. The method of claim 1, wherein the elution buffer comprises glycine at a concentration of no greater than 500 mM, arginine at a concentration of no greater than 100 mM.

5. A method of purifying a polypeptide, the method comprising,
loading the polypeptide onto an affinity chromatography column,
eluting the polypeptide from the affinity chromatography column using an elution buffer,
wherein the elution buffer comprises glycine at a concentration of no greater than 500 mM, and arginine at a concentration of no greater than 100 mM.

6. The method of claim 5, the method comprising,
loading the sample onto an affinity chromatography column,
eluting the polypeptide from the affinity chromatography column using an elution buffer,
wherein the elution buffer comprises glycine and arginine and the glycine and arginine are present at a ratio of at least 1:2 (molar ratio).

7. The method of claim 5, wherein the elution buffer comprises glycine and arginine at a pH between 3.0-5.0, and the glycine and arginine are present at a ratio between 1:2 to 40:1 (molar ratio).

8. The method of claim 7, wherein the elution buffer comprises arginine at a concentration of about 10 to 300 mM and glycine at a concentration of about 50 to 1500 mM.

9. (canceled)

10. The method of claim 1, further comprising a step of adjusting the eluate from the affinity chromatography column to a pH of 4.0 to 9.0.

11. The method of claim 1, further comprising a step of viral inactivation of the eluate from the affinity chromatography column.

12. The method of claim 1, wherein the eluate of the affinity column comprises less than 5% high molecular weight aggregates (HMW).

13. The method of claim 1, wherein the affinity chromatography column uses an antibody or antigen binding fragment that specifically binds the polypeptide.

14. The method of claim 5, further comprising adjusting the concentration of arginine and glycine to in the elution buffer based on the conductivity of a resin for a polypeptide.

15. (canceled)

16. The method of claim 1, comprising subjecting the eluate from the affinity chromatography column to a step of chromatography, wherein the step of chromatography is selected from the group consisting of ion exchange, mixed mode, or hydroxyapatite chromatography.

17-19. (canceled)

20. The method of claim 16, wherein the ion exchange chromatography is anion exchange chromatography or cation exchange chromatography.

21. (canceled)

22. The method of claim 1, comprising determining the conductivity limit of step of chromatography and adjusting the glycine and/or arginine concentration of the affinity chromatography column elution buffer to the conductivity limit, wherein the conductivity limit is about ≤30 mS/cm.

23. (canceled)

24. The method of claim 1, wherein the affinity chromatography column elution buffer has a pH of 3.0-5.0.

25. The method of claim 1, wherein the polypeptide is a recombinant protein and/or an enzyme.

26. (canceled)

27. The method of claim 1, wherein the polypeptide retains specific activity of at least 80% compared to the specific activity before purification.

28. The method of claim 1, wherein the polypeptide is recombinant human arylsulfatase A or recombinant iduronate-2-sulfatase.

29. (canceled)

30. The method of claim 1, wherein the polypeptide does not comprise a domain that binds Protein A or Protein G.

31-32. (canceled)

33. A method of purifying a polypeptide, comprising (a) loading the polypeptide onto an affinity chromatography column, wherein the affinity chromatography column comprises a ligand selected from a library of ligands for specific binding to the polypeptide, (b) eluting the polypeptide from the affinity chromatography column using an elution buffer comprising glycine and arginine, wherein the glycine concentration is X mM, wherein the arginine concentration is Y mM, wherein the concentration of glycine and arginine satisfies an inequality 0.02X+0.1Y≤10, wherein pH of the elution buffer is from 3.0 to 5.0, and (c) subjecting the eluate from the affinity chromatography column to an ion exchange chromatography or mixed-mode chromatography.

34. (canceled)
